(19) United States
(12) Patent Application Publication
(10) Pub. No.: US 2025/0295566 A1
(43) Pub. Date: Sep. 25, 2025

(54) DIESTER HAIR STYLING FORMULATIONS, AND USES THEREOF

(71) Applicant: Chembeau LLC, Santa Barbara, CA (US)

(72) Inventor: Eric D. Pressly, Santa Barbara, CA (US)

(21) Appl. No.: 19/082,061

(22) Filed: Mar. 17, 2025

Related U.S. Application Data
(60) Provisional application No. 63/567,171, filed on Mar. 19, 2024.

(51) Int. Cl. A61K 8/37; A61K 8/23; A61K 8/34; A61K 8/36; A61K 8/42; A61K 8/46; A61Q 5/04

(52) U.S. Cl. CPC A61K 8/37; A61K 8/23; A61K 8/342; A61K 8/361; A61K 8/42; A61K 8/466; A61Q 5/04

(57) ABSTRACT

The present application relates to compounds, formulations, kits and methods of use in the cosmetics field for styling hair (e.g., relaxing, curling, straightening and/or perming hair). A cosmetic formulation comprising a diester compound of Formula (I) or Formula (II), or a combination thereof, a hair treatment agent (e.g., a reducing agent, such as a sulfite salt), and one or more cosmetically acceptable carriers or excipients. The structures of compounds of Formula (I) and Formula (II) are shown below:

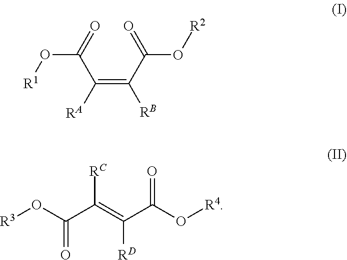

### INCORPORATION BY REFERENCE TO ANY PRIORITY APPLICATIONS

Any and all applications for which a foreign or domestic priority claim is identified in the Application Data Sheet or PCT Request as filed with the present application are hereby incorporated by reference under 37 CFR 1.57, and Rules 4.18 and 20.6. This application claims the benefit of U.S. Provisional Application No. 63/567,171, filed on Mar. 19, 2024, which is incorporated by reference herein in its entirety for all purposes.

### BACKGROUND

### Field

The present application generally relates to cosmetic and personal care formulations, kits and methods of use thereof. Particularly the application is related to cosmetic formulations for hair treatment.

### Description of the Related Art

Hair is a protein filament that grows from follicles found in the dermis. Hair is primarily composed of proteins, notably alpha-keratin. Alpha-keratin is a fibrous structural protein (polypeptide chain), which is made up of amino acids that form a repeating secondary structure such as alpha-helix. These alpha-helix chains of amino acids are typically bound to each other by hydrogen bonds, salt bridges, and/or disulfide bonds.

At alkaline pH (e.g., when lye-based relaxers are used on hair) protein secondary structures within the hair are broken so that straightening of the hair may be achieved. While alkali-based relaxers are effective at relaxing and straightening hair, they may result in a reduction of hair strength and a loss of hair through breakage. Alkaline conditions also damage the hair and may cause the cuticle of the hair strands to become roughened resulting in ruffled, tangled, and generally unmanageable hair that makes hair look lusterless and dull.

Yet another issue with the use of alkali-based hair relaxers is that their application leads to completely relaxed and straightened hair without any method to retain the level of curl. Furthermore, due to the reduction of hair strength and potential loss of hair due to breakage from such relaxing processes, any further application of a permanent wave process (i.e., to introduce a controlled amount of curl) to the already relaxed and straightened hair would result in further damage and/or breakage of the hair.

In addition, traditional perms use thiol or sulfur-containing reducing agents followed by a second oxidizing step, typically using hydrogen peroxide, to set the hair. However, these thiol or sulfur-containing reducing agents are foul smelling and, if not properly rinsed between the steps, can damage and/or break hair. In addition, the use of a second oxidizing step increases the time and complexity of the treatment process.

Furthermore, formaldehyde-free hair straightening products are used by professional beauty salons for smoothing hair, and contain chemicals that release formaldehyde and other chemicals upon activation. However, some formaldehyde-free hair straightening products when used release toxic chemicals that may cause systemic toxicity in a user, such as kidney injury. See Abu-Amer, Nabil, et al. “Acute kidney injury following exposure to formaldehyde-free hair-straightening products.” Case Reports in Nephrology and Dialysis 12.2 (2022): 112-116.

Previous work described in U.S. Pat. No. 9,326,926 utilizes an acidic keratin treatment to reduce hair damages caused by hair treatment, and previous work described in U.S. Pat. No. 9,713,583 utilizes an acidic keratin treatment with maleic acid or a maleate salt (e.g. bis-aminopropyl diglycol dimaleate) to affect the curl of hair. However, the active agents in the keratin treatment are very acidic and may be incompatible with the hair treatment formulations such as hair relaxing and perming formulations, which require a high pH to be effective. As such, careful and proper mixing of the acidic keratin treatment is required to not substantially alter the pH and the performance of the formulations.

As such, there remains a need to develop a convenient, safe and effective hair treatment that can reduce hair damages during hair treatment and improve hair condition.

### SUMMARY

In one aspect, a method of styling hair to achieve a hair style is described. The method includes: applying a cosmetic formulation to the hair; wherein the cosmetic formulation comprises a compound of Formula (I) and a reducing agent comprising a sulfite salt; wherein the amount of compound of Formula (I) in the cosmetic formulation is at least about 1% by weight; and wherein the compound of Formula (I) has the structure:

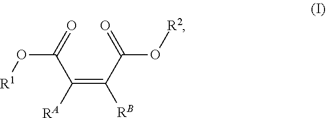

wherein: each of R¹ and R² is independently selected from the group consisting of an optionally substituted C₈ alkyl; and each of R^{A} and R^{B} is hydrogen.

In some embodiments, the method further comprises treating the hair with a styling tool. In some embodiments, the hair style is achieved at most about 30 minutes after the cosmetic formulation is applied to the hair. In some embodiments, the hair style is achieved by the method is retained after the hair is washed 1-50 times. In some embodiments, the hair style is selected from the group consisting of permed hair, curled hair, straightened hair and relaxed hair. In some embodiments, the method is a method of perming hair, comprising applying the cosmetic formulation to the hair and treating the hair with a styling tool. In some embodiments, the method does not comprise applying an oxidizing agent to the hair.

In another aspect, a cosmetic formulation is described. The cosmetic formulation includes: about 0.1-20 wt. % of a sulfite salt; about 0.1-20 wt. % of a surfactant; a cosmetically acceptable carrier; and about 0.1-20 wt. % of a compound of Formula (I), wherein the compound of Formula (I) has the structure:

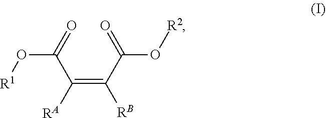

wherein: each of R¹ and R² is independently selected from the group consisting of an optionally substituted C₈ alkyl; and each of R^{A} and R^{B} is hydrogen.

In some embodiments, the compound of Formula (I) is selected from the group consisting of dioctyl maleate and bis(2-ethylhexyl)maleate. In some embodiments, the sulfite salt is sodium sulfite. In some embodiments, the surfactant comprises a compound selected from the group consisting of an anionic surfactant, a cationic surfactant, and a combination thereof. In some embodiments, the cosmetically acceptable carrier is water. In some embodiments, the formulation comprises a pH of about 7.5-10. In some embodiments, the cosmetic formulation further comprises a fragrance.

In another aspect, a method of styling hair is described. The method includes: applying a cosmetic formulation to hair, wherein the cosmetic formulation comprises a compound of Formula (I) and a reducing agent comprising a sulfite salt, wherein the amount of compound of Formula (I) in the cosmetic formulation is at least about 1% by weight, and wherein the compound of Formula (I) has the structure:

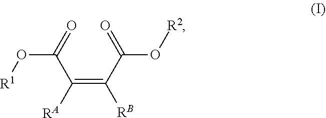

wherein: each of R¹ and R² is independently selected from the group consisting of an optionally substituted C₈ alkyl; and each of R^{A} and R^{B} is hydrogen; and styling the hair; wherein applying the cosmetic formulation and styling the hair achieves a hair style.

In some embodiments, styling the hair comprises treating the hair with a styling tool. In some embodiments, styling the hair comprises contacting the hair with a styling tool. In some embodiments, the styling tool is selected from the group consisting of a brush, a comb, a roller, and combinations thereof. In some embodiments, the hair style is achieved at most about 30 minutes after the cosmetic formulation is applied to the hair. In some embodiments, styling the hair is performed after applying the cosmetic formulation to the hair. In some embodiments, applying the cosmetic formulation to the hair is performed after styling the hair.

In some embodiments, the hair style is substantially retained after washing the hair 1-50 times. In some embodiments, washing the hair is selected from the group consisting of rinsing, shampooing, conditioning, drying, and combinations thereof. In some embodiments, the cosmetic formulation is applied to the hair 1-10 times a month. In some embodiments, the cosmetic formulation is applied to the hair at least 1 time per month. In some embodiments, the method achieves a hair texture selected from the group consisting of increased hair smoothing, decreased hair frizz, improved touch, increased hair combability, increased hair shine, and combinations thereof. In some embodiments, the method provides increased hair smoothing, decreased hair frizz, improved touch, increased hair combability, increased hair shine, or combinations thereof.

In some embodiments, the hair style is selected from the group consisting of permed hair, curled hair, straightened hair and relaxed hair. In some embodiments, the hair style comprises permed hair, curled hair, straightened hair, relaxed hair, or combinations thereof. In some embodiments, the method is a method of perming hair. In some embodiments, the method is a method of perming hair, and wherein styling the hair comprises rolling the hair. In some embodiments, the method is a method of straightening hair. In some embodiments, the method is a method of straightening hair, and wherein styling the hair comprises brushing or combing the hair. In some embodiments, the method does not comprise applying an oxidizing agent to the hair.

In another aspect, a cosmetic formulation is described. The cosmetic formulation includes: about 0.1 wt. % to about 20 wt. % of a sulfite salt; about 0.1 wt. % to about 20 wt. % of a surfactant; a cosmetically acceptable carrier; and about 0.1 wt. % to about 20 wt. % of a compound of Formula (I), wherein the compound of Formula (I) has the structure:

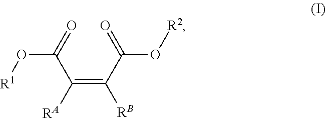

wherein: each of R¹ and R² is independently selected from the group consisting of an optionally substituted C₈ alkyl; and each of R^{A} and R^{B} is hydrogen.

In some embodiments, the compound of Formula (I) is selected from the group consisting of dioctyl maleate and bis(2-ethylhexyl)maleate. In some embodiments, the cosmetic formulation comprises about 3 wt. % to about 18 wt. % of the compound of Formula (I). In some embodiments, the sulfite salt is selected from the group consisting of sodium sulfite, potassium sulfite, and combinations thereof. In some embodiments, the cosmetic formulation comprises about 0.8 wt. % to about 6 wt. % of the sulfite salt.

In some embodiments, the surfactant comprises a surfactant compound selected from the group consisting of an anionic surfactant, a cationic surfactant, a natural surface active agent, and combinations thereof. In some embodiments, the surfactant comprises an anionic surfactant, a cationic surfactant, a natural surface active agent, or combinations thereof. In some embodiments, the surfactant comprises the anionic surfactant and the natural surface active agent. In some embodiments, the natural surface active agent comprises oleic acid. In some embodiments, the natural surface active agent is oleic acid. In some embodiments, the anionic surfactant is selected from the group consisting of sodium cocoyl isethionate, stearamidopropyl dimethyl amine, oleyl alcohol, and combinations thereof. In some embodiments, the cosmetic formulation comprises about 0.5 wt. % to about 5 wt. % of the surfactant.

In some embodiments, the cosmetically acceptable carrier comprises a carrier compound selected from the group consisting of water, ethanol, and combinations thereof. In some embodiments, the cosmetically acceptable carrier comprises water, ethanol, or a combination thereof. In some embodiments, the cosmetic formulation comprises about 70 wt. % to about 95 wt. % of cosmetically acceptable carrier. In some embodiments, the cosmetic formulation has a pH of about 7.5 wt. % to about 10. In some embodiments, cosmetic formulation further comprises a fragrance. In some embodiments, the cosmetic formulation comprises about 0.1 wt. % to about 1 wt. % of the fragrance.

In another aspect, a method of styling hair is described. The method includes: applying a cosmetic formulation to hair, wherein the cosmetic formulation comprises: about 0.8 wt. % to about 6 wt. % of sodium sulfite; about 0.5 wt. % to about 5 wt. % of a surfactant, wherein the surfactant comprises oleic acid, sodium cocoyl isethionate, stearamidopropyl dimethyl amine, oleyl alcohol, or combinations thereof; about 3 wt. % to about 18 wt. % of bis(2-ethylhexyl) maleate; about 0.1 wt. % to about 3 wt. % of ethanol; and a balance of water; wherein the cosmetic formulation is absent of an oxidizing agent; and styling the hair; wherein applying the cosmetic formulation and styling the hair achieves a hair style selected from the group consisting of permed hair, curled hair, straightened hair and relaxed hair, and combinations thereof; and wherein applying the cosmetic formulation and styling the hair provides increased hair smoothing, decreased hair frizz, improved touch, increased hair combability, increased hair shine, or combinations thereof.

### BRIEF DESCRIPTION OF THE DRAWINGS

FIG. 1A shows a back image of a mannequin with a left side of hair treated with a cosmetic formulation and straightened according to some embodiments, and a right side of hair untreated.

FIG. 1B shows a front image of the mannequin of FIG. 1A with a left side of hair treated with a cosmetic formulation and straightened according to some embodiments, and a right side of hair untreated.

FIG. 2A shows an image of a mannequin with hair curled in curlers and a right side of hair treated with a cosmetic formulation, according to some embodiments.

FIG. 2B shows a back image of the mannequin of FIG. 2A with a right side of hair treated with a cosmetic formulation soon after curling, according to some embodiments.

FIG. 2C shows a front image of the mannequin of FIG. 2B with a right side of hair treated with a cosmetic formulation with multiple washes after curling, according to some embodiments.

FIG. 3A shows an image of a bleached hair swatch treated with a cosmetic formulation and curled, according to some embodiments.

FIG. 3B shows an image of a bleached hair swatch treated with a cosmetic formulation and curled, according to some embodiments.

FIG. 3C shows an image of a control bleached hair swatch untreated and not curled.

FIG. 4A shows a front image of a mannequin with a left side of hair thrice treated with a cosmetic formulation and straightened according to some embodiments, and a right side of hair untreated.

FIG. 4B shows a front image of a mannequin with a left side of hair twice treated with a cosmetic formulation and straightened according to some embodiments, and a right side of hair untreated.

FIG. 4C shows a front image of a mannequin with a left side of hair once treated with a cosmetic formulation and straightened according to some embodiments, and a right side of hair untreated.

FIG. 5A shows a back image of a mannequin with a left side of hair treated with a cosmetic formulation and relaxed according to some embodiments, and a right side of hair treated with a control formulation.

FIG. 5B shows a front image of the mannequin of FIG. 5A with a left side of hair treated with a cosmetic formulation and relaxed according to some embodiments, and a right side of hair treated with a control formulation.

### DETAILED DESCRIPTION

The present application relates to cosmetic formulations, kits and methods of use thereof. The cosmetic formulation includes an effective amount of one or more diester compounds of Formula (I) or (II) as described herein, a hair treatment agent (e.g., a reducing agent, such as a sulfite salt), and a cosmetically acceptable carrier or excipient, or a combination thereof. The cosmetic formulation can be used to treat and/or style hair. For example, the cosmetic formulation can be used to treat hair or prevent damage to hair that is caused by hair treatments such as styling (e.g., curling, relaxing, straightening and/or perming hair).

In contrast to previously reported keratin treatments that contain acidic active agents, the diester compounds described herein are relatively pH neutral. As such, cosmetic formulations containing the diester compound(s) described herein may be liberally applied without affecting the effectiveness of high pH cosmetic co-treatments, such as hair styling (e.g., relaxing, curling, straightening and/or perming), which require careful balance of pH for adequate performance and effectiveness. One main advantage of the cosmetic formulations described herein is that the amount of the pH neutral diester compounds used in the cosmetic treatment can be in a wide range without concern to the relative amount of other pH sensitive treatments, such as hair styling (e.g., hair relaxing, hair curling, hair straightening, hair perming or hair curling). Furthermore, in some embodiments, cosmetic formulations described herein may be utilized in a relatively quick single step to style (e.g., relax, straighten, curl and/or perm) without the use of an oxidizing agent (e.g., a peroxide), which is typically utilized in a second step of some hair treatments (e.g., perming). Furthermore, in some embodiments, cosmetic formulations described herein may be utilized without, without substantially, without exposing or without substantially exposing the user to toxic chemicals, such as formaldehyde and/or products of formaldehyde producing compounds (e.g., glyoxyloyl carbocysteine).

In some embodiments, the cosmetic formulation styles or aids in styling hair by changing the texture of hair (e.g., touch, smoothing the hair texture, decreasing and/or managing hair frizz, combability of the hair, shine of the hair). In some embodiments, the cosmetic formulation relaxes (e.g., straightens), curls and/or perms hair. In some embodiments, the cosmetic formulation styles and/or changes the texture of hair permanently or semi-permanently.

### Definitions

Unless defined otherwise, all technical and scientific terms used herein have the same meaning as is commonly understood by one of ordinary skill in the art.

The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where a range of values is provided, it is understood that the upper and lower limit, and each intervening value between the upper and lower limit of the range is encompassed within the embodiments.

As used herein, the term “wt. %” refers to percentage by weight.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₂₂” includes C₁, C₂, C₃, C₄, C₅, C₆, C₇, C₈, C₉, C₁₀, C₁₁, C₁₂, up to C₂₂, and a range defined by any of the two numbers.

As used herein, “alkyl” refers to a straight or branched hydrocarbon chain that is fully saturated (i.e., contains no double or triple bonds). The alkyl group may have 1 to 22 carbon atoms (whenever it appears herein, a numerical range such as “1 to 22” refers to each integer in the given range; e.g., “1 to 22 carbon atoms” means that the alkyl group may consist of 1 carbon atom, 2 carbon atoms, 3 carbon atoms, etc., up to and including 22 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula-OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 22 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2, -dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 22 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “cycloalkyl” means a fully saturated carbocyclyl ring or ring system. Examples include cyclopropyl, cyclobutyl, cyclopentyl, and cyclohexyl.

As used herein, “alkoxyalkyl” or “(alkoxy)alkyl” refers to an alkoxy group connected via an alkylene group, such as C₂-C₈ alkoxyalkyl, or (C₁-C₆ alkoxy) C₁-C₆ alkyl, for example, —(CH₂)₁₋₃—OCH₃.

When a group is described as “optionally substituted” it may be either unsubstituted or substituted. Likewise, when a group is described as being “substituted”, the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl) C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl) C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy) C₁-C₆ alkyl, —O(C₁-C₆ alkoxy) C₁-C₆ alkyl; (C₁-C₆ haloalkoxy) C₁-C₆ alkyl; —O(C₁-C₆ haloalkoxy) C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfino, —OSO₂C₁₋₄alkyl, and oxo (═O). Wherever a group is described as “optionally substituted” that group can be substituted with the above substituents.

The term “hair” refers to one or more than one strands of hair, as well as the natural components of hair, such as oil from a body. Hair may refer to virgin hair or processed hair, for example hair that has been exposed to hair waving or hair straightening formulations.

The term “untreated hair” refers to virgin hair, or refers to hair that has been treated with a cosmetic or hair treatment formulation (e.g. a bleaching or a coloring formulation) that does not contain one or more diester compounds described herein (e.g. a compound of Formula (I) or Formula (II)).

The term “shampoo” generally refers to a liquid or semi-solid formulation applied to the hair that contains detergent or soap for washing the hair.

The term “conditioner” generally refers to a formulation (e.g., liquid, cream, lotion, gel, semi-solid) applied to the hair to soften the hair, smooth the hair, and/or change the sheen of the hair.

### Diester Compounds

Some embodiments described herein generally relate to a compound of Formula (I) or Formula (II), or a combination thereof, having the structure:

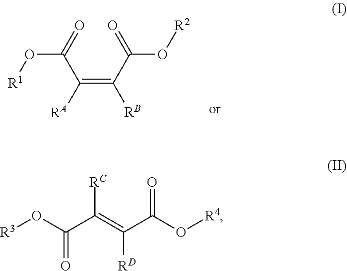

wherein: each of R¹, R², R³ and R⁴ is independently selected from the group consisting of an optionally substituted C₁-C₂₂ alkyl, an optionally substituted C₂-C₂₂ alkenyl, an optionally substituted C₂-C₂₂ alkynyl, an optionally substituted C₃-C₁₀ carbocyclyl, an optionally substituted C₆-C₁₀ aryl, an optionally substituted 5 to 10 membered heteroaryl, an optionally substituted 3 to 10 membered heterocyclyl, and an optionally substituted C₂-C₂₂ alkoxyalkyl; and each of R^{A}, R^{B}, R^{C} and R^{D} is independently hydrogen or an optionally substituted C₁-C₂₂ alkyl.

In some embodiments, the compound can be a compound of Formula (I), where each of R¹ and R² is independently selected from the group consisting of an optionally substituted C₁-C₂₂ alkyl, an optionally substituted C₂-C₂₂ alkenyl, an optionally substituted C₂-C₂₂ alkynyl, an optionally substituted C₃-C₁₀ carbocyclyl, an optionally substituted C₆-C₁₀ aryl, an optionally substituted 5 to 10 membered heteroaryl, an optionally substituted 3 to 10 membered heterocyclyl, and an optionally substituted C₂-C₂₂ alkoxyalkyl; and each of R^{A} and R^{B} is independently hydrogen or an optionally substituted C₁-C₂₂ alkyl. For example, in some embodiments, each of R¹ and R² is independently selected from the group consisting of an optionally substituted C₈ alkyl; and each of R^{A} and R^{B} is hydrogen.

In some embodiments of the compound of Formula (I), each of R¹ and R² is independently an unsubstituted C₁-C₂₂ alkyl (e.g., unsubstituted C₁-C₆ alkyl or unsubstituted C₇. C₁₂ alkyl). In some embodiments, each of R¹ and R² is independently an unsubstituted C₂-C₂₂ alkenyl (e.g., unsubstituted C₂-C₆ alkenyl or unsubstituted C₇-C₁₂ alkenyl). In some embodiments, each of R¹ and R² is independently an unsubstituted C₂-C₂₂ alkynyl (e.g., unsubstituted C₂-C₆ alkynyl or unsubstituted C₇-C₁₂ alkynyl). In some embodiments, each of R¹ and R² is independently an unsubstituted C₃-C₁₀ carbocyclyl (e.g., unsubstituted C₃-C₆ cycloalkyl). In some embodiments, each of R¹ and R² is independently an unsubstituted C₆-C₁₀ aryl (e.g., unsubstituted phenyl). In some embodiments, each of R¹ and R² is independently an unsubstituted 5 to 10 membered heteroaryl (e.g., unsubstituted 5 or 6 membered heteroaryl containing 1 to 3 heteroatoms selected from the group consisting of N, O and S). In some embodiments, each of R¹ and R² is independently an unsubstituted 3 to 10 membered heterocyclyl (e.g., unsubstituted 3 to 6 membered heterocyclyl containing 1 to 3 heteroatoms selected from the group consisting of N, O and S). In some embodiments, each of R¹ and R² is independently an unsubstituted C₂-C₂₂ alkoxyalkyl (e.g., unsubstituted (C₁-C₆ alkoxy) C₁-C₆ alkyl). In some embodiments, R¹ is a substituted C₁-C₂₂ alkyl. In some embodiments, each of R¹ and R² is independently a substituted C₂-C₂₂ alkenyl. In some embodiments, each of R¹ and R² is independently a substituted C₂-C₂₂ alkynyl. In some embodiments, each of R¹ and R² is independently a substituted C₃-C₁₀ carbocyclyl. In some embodiments, each of R¹ and R² is independently a substituted C₆-C₁₀ aryl. In some embodiments, each of R¹ and R² is independently a substituted 5 to 10 membered heteroaryl. In some embodiments, each of R¹ and R² is independently a substituted 3 to 10 membered heterocyclyl. In some embodiments, each of R¹ and R² is independently a substituted C₂-C₂₂ alkoxyalkyl.

In some embodiments of the compound of Formula (I), R^{A} is hydrogen. In some embodiments, R^{A} is an unsubstituted C₁-C₂₂ alkyl. In some embodiments, R^{A} is a substituted C₁-C₂₂ alkyl. In some embodiments, R^{B} is hydrogen. In some embodiments, R^{B} is an unsubstituted C₁-C₂₂ alkyl. In some embodiments, R^{B} is a substituted C₁-C₂₂ alkyl.

In some further embodiments of the compound of Formula (I), each of R¹ and R² is independently an unsubstituted C₁-C₆ alkyl (e.g., methyl, ethyl, propyl, isopropyl, n-butyl, isobutyl, t-butyl, n-pentyl, isopentyl, n-hexyl, isohexyl), and each of R^{A} and R^{B} is hydrogen. In one embodiment, each of R¹ and R² is n-butyl. In some other embodiments, each of R¹ and R² is independently an unsubstituted C₇-C₁₂ alkyl (e.g., n-heptyl, iso-heptyl, n-octyl, iso-octyl, ethylhexyl, 2-ethylhexyl, n-nonyl, isononyl, n-decyl, iso-decyl, ethyloctyl), and each of R^{A} and R^{B} is hydrogen. In some additional embodiments of the compound of Formula (I), each of R¹ and R² is independently an optionally substituted C₈ alkyl, and each of R^{A} and R^{B} is hydrogen. In one embodiment, each of R¹ and R² is n-octyl, and the compound of Formula (I) is dioctyl maleate. In another embodiment, each of R¹ and R² is ethylhexyl (e.g., 2-ethylhexyl), and the compound of Formula (I) is bis(2-ethylhexyl)maleate.

In some embodiments, the compound can be a compound of Formula (II), where each of R³ and R⁴ is independently selected from the group consisting of an optionally substituted C₁-C₂₂ alkyl, an optionally substituted C₂-C₂₂ alkenyl, an optionally substituted C₂-C₂₂ alkynyl, an optionally substituted C₃-C₁₀ carbocyclyl, an optionally substituted C₆-C₁₀ aryl, an optionally substituted 5 to 10 membered heteroaryl, an optionally substituted 3 to 10 membered heterocyclyl, and an optionally substituted C₂-C₂₂ alkoxyalkyl; and each of R^{C} and R^{D} is independently hydrogen or an optionally substituted C₁-C₂₂ alkyl.

In some embodiments of the compound of Formula (II), each of R³ and R⁴ is independently an unsubstituted C₁-C₂₂ alkyl (e.g., unsubstituted C₁-C₆ alkyl or unsubstituted C₇-C₁₂ alkyl). In some embodiments, each of R³ and R⁴ is independently an unsubstituted C₂-C₂₂ alkenyl (e.g., unsubstituted C₂-C₆ alkenyl or unsubstituted C₇-C₁₂ alkenyl). In some embodiments, each of R³ and R⁴ is independently an unsubstituted C₂-C₂₂ alkynyl (e.g., unsubstituted C₂-C₆ alkynyl or unsubstituted C₇-C₁₂ alkynyl). In some embodiments, each of R³ and R⁴ is independently an unsubstituted C₃-C₁₀ carbocyclyl (e.g., unsubstituted C₃-C₆ cycloalkyl). In some embodiments, each of R³ and R⁴ is independently an unsubstituted C₆-C₁₀ aryl (e.g., unsubstituted phenyl). In some embodiments, each of R³ and R⁴ is independently an unsubstituted 5 to 10 membered heteroaryl (e.g., unsubstituted 5 or 6 membered heteroaryl containing 1 to 3 heteroatoms selected from the group consisting of N, O and S). In some embodiments, each of R³ and R⁴ is independently an unsubstituted 3 to 10 membered heterocyclyl (e.g., unsubstituted 3 to 6 membered heterocyclyl containing 1 to 3 heteroatoms selected from the group consisting of N, O and S). In some embodiments, each of R³ and R⁴ is independently an unsubstituted C₂-C₂₂ alkoxyalkyl (e.g., unsubstituted C₁-C₆ alkoxy) C₁-C₆ alkyl). In some embodiments, each of R³ and R⁴ is independently a substituted C₁-C₂₂ alkyl. In some embodiments, each of R³ and R⁴ is independently a substituted C₂-C₂₂ alkenyl. In some embodiments, each of R³ and R⁴ is independently a substituted C₂-C₂₂ alkynyl. In some embodiments, each of R³ and R⁴ is independently a substituted C₃-C₁₀ carbocyclyl. In some embodiments, each of R³ and R⁴ is independently a substituted C₆-C₁₀ aryl. In some embodiments, each of R³ and R⁴ is independently a substituted 5 to 10 membered heteroaryl. In some embodiments, each of R³ and R⁴ is independently a substituted 3 to 10 membered heterocyclyl. In some embodiments, each of R³ and R⁴ is independently a substituted C₂-C₂₂ alkoxyalkyl.

In some embodiments of the compound of Formula (II), R^{C} is hydrogen. In some embodiments, R^{C} is an unsubstituted C₁-C₂₂ alkyl. In some embodiments, R^{C} is a substituted C₁-C₂₂ alkyl. In some embodiments, R^{D} is hydrogen. In some embodiments, R^{D} is an unsubstituted C₁-C₂₂ alkyl. In some embodiments, R^{D} is a substituted C₁-C₂₂ alkyl.

In some further embodiments of the compound of Formula (II), each of R³ and R⁴ is independently an unsubstituted C₁-C₆ alkyl (e.g., methyl, ethyl, propyl, isopropyl, n-butyl, isobutyl, t-butyl, n-pentyl, isopentyl, n-hexyl, isohexyl), and each of R^{C} and R^{D} is hydrogen. In one embodiment, each of R¹ and R² is n-butyl. In some further embodiments, each of R³ and R⁴ is independently an unsubstituted C₇-C₁₂ alkyl (e.g., n-heptyl, iso-heptyl, n-octyl, iso-octyl, ethylhexyl, 2-ethylhexyl, n-nonyl, isononyl, n-decyl, iso-decyl, ethyloctyl), and each of R^{C} and R^{D} is hydrogen. In some additional embodiments, each of R³ and R⁴ is independently an optionally substituted C₈ alkyl, and each of R^{C} and R^{D} is hydrogen. In one embodiment, each of R³ and R⁴ is n-octyl. In another embodiment, each of R³ and R⁴ is ethylhexyl (e.g., 2-ethylhexyl).

In some embodiments, the compound can be dioctyl maleate and/or bis(2-ethylhexyl)maleate.

### Cosmetic Formulations

Some embodiments described herein relate to a cosmetic formulation that can include an effective amount of one or more compounds described herein, for example a compound of Formulae (I) or Formula (II) or a combination thereof (i.e., the active ingredient) as described herein, and one or more cosmetically acceptable carriers or excipients. In some embodiments, the cosmetic formulation is a hair treatment formulation. In some embodiments, a hair treatment formulation is combined with additional ingredients or formulation components to form the cosmetic formulation.

In some embodiments, the cosmetic formulation or hair treatment formulation includes, includes about, includes at least, or includes at least about, 0.05 wt. %, 0.1 wt. %, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. %, 18 wt. %, 20 wt. %, 25 wt. %, 30 wt. %, 35 wt. %, 40 wt. %, 45 wt. %, 50 wt. %, 60 wt. %, 65 wt. %, 70 wt. %, or 75 wt. % of each or total of the one or more compounds of Formula (I) or (II), or a range defined by any two of the preceding values. In some embodiments, the cosmetic formulation or hair treatment formulation includes, includes about, includes at least, or includes at least about, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. %, 18 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. % or 80 wt. % of each or total of the one or more compounds of Formula (I) or (II), or a range defined by any two of the preceding values, when applied to coarse hair (e.g. African American hair) and/or hair subjected to repeated damage caused by bleaching, coloring, perming, relaxing, straightening, heat styling, etc.).

In some embodiments, cosmetically acceptable carrier includes water, ethyl acetate, acetone, an alcohol (e.g., ethanol), polyol, oil, silicone, liposomes, or ester, or combinations thereof. In some embodiments, the polyol is glycol, such as ethylene glycol or propylene glycol. In some embodiments, the water is purified water, distilled water, deionized water, or combinations thereof. In some embodiments, the cosmetic formulation or hair treatment formulation includes, includes about, includes at least, or includes at least about, 0.1 wt. %, 0.2 wt. %, 0.3 wt. %, 0.4 wt. %, 0.5 wt. %, 0.7 wt. %, 1 wt. %, 1.5 wt. %, 2 wt. %, 3 wt. %, 4 wt. %, 5 wt. %, 10 wt. %, 15 wt. %, 20 wt. %, 25 wt. %, 30 wt. %, 35 wt. %, 40 wt. %, 45 wt. %, 50 wt. %, 60 wt. %, 65 wt. %, 70 wt. %, 75 wt. %, 80 wt. %, 85 wt. %, 90 wt. %, 95 wt. %, 98 wt. %, 99 wt. % or a balance of each or total of the cosmetically acceptable carrier, or a range defined by any two of the preceding values. In some embodiments, the cosmetically acceptable carrier includes water and/or ethanol. In some embodiments, the cosmetic formulation or hair treatment formulation includes a balance of the cosmetically acceptable carrier, or a balance of the cosmetically acceptable carrier and incidental impurities.

In some embodiments, the cosmetically acceptable excipient includes a surfactant, a vitamin, a natural extract, a preservative, an antioxidant, a chelating agent, a protein, an amino acid, a humectant, a fragrance, an emollient, a penetrant, a thickener, a viscosity modifier, a hair fixative, a film former, an emulsifier, an opacifying agent, a propellant, a carrier, a salt, a buffer, an anti-static agent, an anti-frizz agent, an anti-dandruff agent, or a colorant, or combinations thereof. In some embodiments, the cosmetically acceptable excipient is typically present in an amount ranging from about 10 wt. % to about 99.99 wt. % of the cosmetic formulation, for example, from about 40 wt. % to about 99 wt. %, or from about 80 wt. % to about to about 99 wt. %. In some embodiments, the cosmetic formulation or hair treatment formulation includes, includes about, includes at least, or includes at least about, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. %, 20 wt. %, 25 wt. %, 30 wt. %, 35 wt. %, 40 wt. %, 45 wt. %, 50 wt. %, 60 wt. %, 65 wt. %, 70 wt. %, or 75 wt. % of each or total of the cosmetically acceptable excipient (e.g., surfactant), or any range of values therebetween.

In some embodiments, the cosmetic formulation is in the form of a gel, liquid (e.g., non-viscous liquid), cream, powder, lotion, milk, mousse, spray, shampoo, conditioner, and the like. For example, in some embodiments, the cosmetic formulation is a non-viscous liquid that can be applied to the hair from a narrow tipped applicator bottle allowing controlled application of the cosmetic formulation.

In some embodiments, a formulation component (e.g., active agent, hair treatment agent) and/or a formulation (e.g., first formulation, second formulation, active agent formulation, hair treatment formulation) are in the form of a pill (e.g., a tablet or capsule). In some embodiments, the pill is configured to be combined with (e.g., dissolved within) another cosmetic formulation component (e.g., a cosmetically acceptable carrier, such as water) prior to application to the hair.

In some embodiments, the cosmetic formulation further includes a pH modifying agent. In some embodiments, the cosmetic formulation further includes one or more hair treatment agents. In some embodiments, the one or more hair treatment agents includes a styling agent (e.g., a relaxing agent, a reducing agent, or combinations thereof). In some embodiments, a relaxing agent includes a compound (e.g., a base) that permanently or substantially permanently breaks bonds (e.g., disulfide bonds) within hair. In contrast, in some embodiments, a hair reducing agent includes a compound (e.g., a sulfur containing compound) that breaks bonds (e.g., disulfide bonds) within hair such that the bonds may be reformed or substantially reformed (e.g., using an oxidizing agent, or using a compound of Formula (I) or (II)). In some embodiments, the relaxing agent is selected from sodium hydroxide, lithium hydroxide, potassium hydroxide, guanidinium hydroxide, ammonium hydroxide, or combinations thereof. In some embodiments, a reducing agent is selected from a sulfite salt, a thioglycolate salt, thioglycolic acid, thiolactic acid, dihydrolipoate, thioglycerol, mercaptopropionic acid, sodium bisulfite, ammonium bisulfide, zinc formaldehyde sulfoxylate, sodium formaldehyde sulfoxylate, sodium metabisulfite, potassium borohydride, pegylated thiols and hydroquinone, and combinations thereof. In some embodiments, the reducing agent is a sulfite salt reducing agent. In some embodiments, the sulfite salt or sulfite reducing agent is sodium sulfite (Na₂SO₃) or potassium sulfite (K₂SO₃), or a combination thereof. In some embodiments, the cosmetic formulation or hair treatment formulation includes, includes about, includes at most, or includes at most about, 0.1 wt. %, 0.5 wt. %, 0.8 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. % or 20 wt. % of each or total of the styling agent (e.g., a relaxing agent, a reducing agent, or combinations thereof), or any range of value therebetween.

In some embodiments, the cosmetic formulation does not or does not substantially include a coloring agent and/or a dye (e.g. an oxidative dye and/or a direct dye). In some embodiments, the cosmetic formulation further includes a product colorant or pigment (e.g. food color) for the purpose of modifying the aesthetic color of the cosmetic formulation.

A particular embodiment of the cosmetic formulation described herein is a hair styling (e.g., hair relaxing, hair curling, hair straightening and/or perming) formulation. In some embodiments, the cosmetic formulation or hair treatment formulation includes a reducing agent. In some embodiments, the cosmetic formulation or hair treatment formulation includes a sulfite reducing agent and a compound of Formula (I) or (II), or a combination thereof. In some embodiments, the cosmetic formulation or hair treatment formulation includes a sulfite reducing agent, a surfactant, a cosmetically acceptable carrier, and a compound of Formula (I) or (II), or a combination thereof. In some embodiments, the cosmetic formulation or hair treatment formulation includes about 0.1-20 wt. % of a sulfite reducing agent, about 0.1-20 wt. % of a surfactant, a cosmetically acceptable carrier, and about 0.1-20 wt. % of a compound of Formula (I) or (II), or a combination thereof. In some further embodiments, the cosmetic formulation or hair treatment formulation includes a compound of Formula (I), where each of R¹ and R² is independently selected from the group consisting of an optionally substituted C₈ alkyl, and each of R^{A} and R^{B} is hydrogen. In further embodiments, the compound of Formula (I) is dioctyl maleate or bis(2-ethylhexyl)maleate, or a combination thereof. The cometic formulation may also include one or more cosmetically acceptable excipients and carrier as described herein.

### Humectants

Humectant is a substance that attracts and retains moisture. Humectants used in cosmetic and personal care products and compositions include synthetic humectants (such as propylene glycol, urea, polyethylene glycol (PEG) and natural humectants (such as sugars, betains, amino acids, honey, aloe extract, hyaluronic acid, and glycerin. In one particular example, the humectant may include glycine betaine (Beta vulgaris), which is a beet sugar extract. Unlike traditional humectants, which draw moisture from the environment, glycine betaine adds hydration directly due to its abundant source of saccharides. One or more humectants may be included in the cosmetic or personal care formulation described herein, in an amount ranging from about 0.1 wt. % to about 15% wt. the formulation, or from about 0.5% wt. to about 10% wt. of the formulation, or from about 1% to about 5% of the formulation. In some embodiments, the cosmetic formulation or hair treatment formulation includes, includes about, includes at most, or includes at most about, 0.1 wt. %, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. % or 20 wt. % of each or total of the humectant, or any range of value therebetween.

### Emollients

Emollient is a substance that protects against wetness or irritation, softens, soothes, coats, lubricates, moisturizes, protects, and/or cleanses the skin. Suitable emollients for use in the cosmetic formulations include, but are not limited to, a silicone compound (e.g., dimethicone, cyclomethicone, dimethicone copolyol or a mixture of cyclopentasiloxane and dimethicone/vinyldimethicone cross polymer, cyclopentasiloxane polysilicone), polyols such as sorbitol, glycerin, propylene glycol, ethylene glycol, polyethylene glycol, caprylyl glycol, polypropylene glycol, 1,3-butane diol, hexylene glycol, isoprene glycol, xylitol; ethylhexyl palmitate; a triglyceride such as caprylic/capric triglyceride and fatty acid ester such as cetearyl isononanoate or cetyl palmitate. In a specific embodiment, the emollient is dimethicone, amidodimethicone, dimethiconol, cyclopentasiloxane, potassium dimethicone PEG-7 panthenyl phosphate, or combinations thereof. More than one emollient may be included in the cosmetic or personal care formulation described herein, in an amount ranging from about 0.1 wt. % to about 15% wt. the formulation, or from about 0.5% wt. to about 10% wt. of the formulation, or from about 1% to about 5% of the formulation. In some embodiments, the cosmetic formulation or hair treatment formulation includes, includes about, includes at most, or includes at most about, 0.1 wt. %, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. % or 20 wt. % of each or total of the emollient, or any range of values therebetween.

### Emulsifiers

The formulation may also contain one or more emulsifiers. Suitable emulsifiers include, but are not limited to, copolymers of an unsaturated ester and styrene sulfonate monomer, cetearyl alcohol, glyceryl ester, polyoxyethylene glycol ether of cetearyl alcohol, stearic acid, polysorbate-20, ceteareth-20, lecithin, glycol stearate, polysorbate-60, polysorbate-80, or combinations thereof. More than one emulsifier may be included in the formulation in the cosmetic or personal care formulation described herein, in an amount ranging from about 0.05 wt. % to about 15% wt. the formulation, or from about 0.1% wt. to about 10% wt. of the formulation, or from about 0.5% to about 5% of the formulation. In some embodiments, the cosmetic formulation or hair treatment formulation includes, includes about, includes at most, or includes at most about, 0.1 wt. %, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. % or 20 wt. % of each or total of the emulsifier, or any range of values therebetween.

### Surfactants and Solubilizers

In some embodiments, the cosmetic or personal care formulations may further include one or more surfactants, or solubilizer, or combinations thereof.

Solubilizers, as used herein, refers to a compound that can help to make otherwise insoluble liquids soluble in water. For example, essential oils do not mix well with water. Mixing essential oils with a solubilizer before adding to water will help the essential oil mix well with water. Examples of solubilizers include polysorbates, safflower oleosomes, and propanediol.

Surfactants are surface-active agents that are able to reduce the surface tension of water and cause the cosmetic formulation or hair treatment formulation to slip across or onto the skin or hair. Surfactants also include detergents and soap. The surfactants may be amphoteric, anionic, cationic, a natural surface active agent, or combinations thereof. In some embodiments, Suitable surfactants that may be used in the formulation include, but are not limited to, 3-aminopropane sulfonic acid, almond amide, almond amidopropyl betaine, almond amidopropylamine oxide, aluminum hydrogenated tallow glutamate, aluminum lanolate, aminoethyl sulfate, aminopropyl lauryl glutamine, ammonium C12-15 alkyl sulfate, ammonium C12-15 pareth C12-16 sulfate, ammonium alkyl sulfate, ammonium C9-10 perfluoroalkylsulfonate, ammonium capryleth sulfate, ammonium capryleth-3 sulfate, ammonium monoglyceride sulfate, ammonium sulfate, ammonium isothionate, ammonium isethionate, sodium cocoyl isethionate, ammonium cocoyl sarcosinate, ammonium cumene sulfonate, ammonium dimethicone copolyol sulfate, ammonium dodecylbenzenesulfonate, ammonium isostearate, ammonium laureth sulfate, ammonium laureth-12 sulfate, ammonium laureth-5 sulfate, ammonium laureth-6 carboxylate, ammonium laureth-7 sulfate, ammonium laureth-8 carboxylate, ammonium laureth-9 sulfate, ammonium lauroyl sarcosinate, ammonium lauryl sulfate, ammonium lauryl sulfosuccinate, ammonium myreth sulfate, ammonium myristyl sulfate, ammonium nonoxynol-30 sulfate, ammonium nonoxynol-4 sulfate, ammonium oleate, ammonium palm kernel sulfate, ammonium polyacrylate, ammonium stearate, ammonium tallate, ammonium xylene sulfonate, amp-isostearoyl gelatin/keratin amino acids/lysine hydroxypropyltrimonium chloride, amp-isostearoyl hydrolyzed collagen, apricot kernel oil PEG-6 esters, apricot amide, apricot amidopropyl betaine, arachideth-20, avocadamide, avocadamidopropyl betaine, babassuamide, babassuamidopropyl betaine, babassuamidopropylamine oxide, behenalkonium chloride, behenamide, behenamidopropyl betaine, behenamine oxide, sodium laureth sulfate, sodium lauryl sulfate, a polyoxyether of lauryl alcohol or ceteareth-20, or combinations thereof. In some embodiments, Suitable surfactants that may be used in the formulation include, but are not limited to, 3-aminopropane sulfonic acid, almond amide, almond amidopropyl betaine, almond amidopropylamine oxide, aluminum hydrogenated tallow glutamate, aluminum lanolate, aminoethyl sulfate, aminopropyl lauryl glutamine, ammonium C12-15 alkyl sulfate, ammonium C12-15 pareth sulfate, ammonium C12-16 alkyl sulfate, ammonium C9-10 perfluoroalkylsulfonate, ammonium capryleth sulfate, ammonium capryleth-3 sulfate, ammonium monoglyceride sulfate, ammonium sulfate, ammonium isothionate, ammonium isethionate, oleic acid, sodium cocoyl isethionate, ammonium cocoyl sarcosinate, ammonium cumene sulfonate, ammonium dimethicone copolyol sulfate, ammonium dodecylbenzenesulfonate, ammonium isostearate, ammonium laureth sulfate, ammonium laureth-12 sulfate, ammonium laureth-5 sulfate, ammonium laureth-6 carboxylate, ammonium laureth-7 sulfate, ammonium laureth-8 carboxylate, ammonium laureth-9 sulfate, ammonium lauroyl sarcosinate, ammonium lauryl sulfate, ammonium lauryl sulfosuccinate, ammonium myreth sulfate, ammonium myristyl sulfate, ammonium nonoxynol-30 sulfate, ammonium nonoxynol-4 sulfate, ammonium oleate, ammonium palm kernel sulfate, ammonium polyacrylate, ammonium stearate, ammonium tallate, ammonium xylene sulfonate, amp-isostearoyl gelatin/keratin amino acids/lysine hydroxypropyltrimonium chloride, amp-isostearoyl hydrolyzed collagen, apricot kernel oil PEG-6 esters, apricot amide, apricot amidopropyl betaine, arachideth-20, avocadamide, avocadamidopropyl betaine, babassuamide, babassuamidopropyl betaine, babassuamidopropylamine oxide, behenalkonium chloride, behenamide, behenamidopropyl betaine, behenamine oxide, sodium laureth sulfate, a polyoxyether of lauryl alcohol or ceteareth-20, sodium, potassium, ammonium of long chain alkyl sulfonates and alkyl aryl sulfonates, such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; alkyl sulfates, such as sodium lauryl sulfate; and coconut based anions, such as sodium methyl cocoyl laurate, quaternary ammonium compounds, such as benzalkonium chloride, benzethonium chloride, behentrimonium chloride, behentrimonium methosulfate, cetrimonium bromide, stearyl dimethylbenzyl ammonium chloride, stearamidopropyl dimethyl amine, protonated amines of stearamidopropyl dimethyl amine, and a coconut amine (e.g., cocamidopropyl dimethylamine), ethylene glycol monostearate, propylene glycol myristate, glyceryl monostearate, glyceryl stearate, polyglyceryl-4-oleate, oleyl alcohol, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide, N-dodecyl-β-alanine, sodium N-lauryl-β-iminodipropionate, myristoamphoacetate, lauryl betaine and lauryl sulfobetaine, saponin, lecithin, soybean phospholipid, hydrogenated soybean phospholipid, soybean lysophospholipid, hydrogenated soybean lysophospholipid, egg yolk lecithin, hydrogenated egg yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid, mannosyl erythritol lipid, surfactin, salts thereof, or combinations thereof.

Suitable anionic surfactants include, but are not limited to, those containing carboxylate, sulfonate and sulfate ions. Examples of anionic surfactants include oleic acid, sodium cocoyl isethionate, sodium, potassium, ammonium of long chain alkyl sulfonates and alkyl aryl sulfonates, such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; alkyl sulfates, such as sodium lauryl sulfate; and coconut based anions, such as sodium methyl cocoyl laurate, and salts thereof. Examples of cationic surfactants include, but are not limited to, quaternary ammonium compounds, such as benzalkonium chloride, benzethonium chloride, behentrimonium chloride, behentrimonium methosulfate, cetrimonium bromide, stearyl dimethylbenzyl ammonium chloride, stearamidopropyl dimethyl amine, protonated amines of stearamidopropyl dimethyl amine, and a coconut amine (e.g., cocamidopropyl dimethylamine), and salts thereof. Examples of nonionic surfactants include ethylene glycol monostearate, propylene glycol myristate, glyceryl monostearate, glyceryl stearate, polyglyceryl-4-oleate, oleyl alcohol, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide, and salts thereof. Examples of amphoteric surfactants include sodium N-dodecyl-β-alanine, sodium N-lauryl-β-iminodipropionate, myristoamphoacetate, lauryl betaine and lauryl sulfobetaine, and salts thereof.

In some embodiments, the surfactants that may be used are natural surface active agents, such as oleic acid, saponin, lecithin, soybean phospholipid, hydrogenated soybean phospholipid, soybean lysophospholipid, hydrogenated soybean lysophospholipid, egg yolk lecithin, hydrogenated egg yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid, mannosyl erythritol lipid, surfactin and salts thereof.

More than one surfactant may be included in the formulation. In some embodiments, the cosmetic or personal care formulation described herein includes one or more surfactants in an amount ranging from about 0.1 wt. % to about 15% wt. the formulation, or from about 0.5% wt. to about 10% wt. of the formulation, or from about 1% to about 5% of the formulation. In some embodiments, the cosmetic formulation or hair treatment formulation includes, includes about, includes at most, or includes at most about, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. % or 20 wt. % of each or total of the surfactant, or any range of values therebetween.

### Essential Oils

Various essential oils may be used in the cosmetic or personal care formulations described herein. Essential oils may act as a fragrance, or may also confer additional benefits due to the nature of the essential oils, including but not limited to cleansing, nourishing, and strengthening the hair follicles and shaft. However, most essential oils are highly concentrated and potent, and they may have adverse effect on skin if used undiluted. In certain hair and skin care products, essential oils are diluted with carrier oils. Carrier oils are a vegetable origin extracted from nuts and seeds by cold pressing. Carrier oils provide lubrication and moisture and help with the absorption of essential oils into the skin. In some embodiments of the cosmetic formulation described herein, the one or more essential oils are used without any carrier oil. In some embodiments, the cosmetic formulation or hair treatment formulation includes about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, or 5% by weight of each or total of the essential oils, or a range defined by any two preceding values.

Non-limiting embodiments of the essential oils that may be used are described herein. In some embodiments, the one or more essential oils are selected from the group consisting of lavender oil, tea tree oil, peppermint oil, rosemary oil, and combinations thereof. Other essential oils that may be used include, but are not limited to, allspice berry essential oil, angelica seed essential oil, anise seed essential oil, basil essential oil, bay laurel essential oil, bay essential oil, bergamot essential oil, blood orange essential oil, camphor essential oil, caraway seed essential oil, cardamom seed essential oil, carrot seed essential oil, cassia essential oil, catnip essential oil, cedarwood essential oil, celery seed essential oil, chamomile essential oil, chamomile roman essential oil, cinnamon bark essential oil, cinnamon leaf essential oil, citronella essential oil, clary sage essential oil, clove bud essential oil, coriander seed essential oil, cypress essential oil, elemi essential oil, eucalyptus essential oil, fennel essential oil, fir needle essential oil, frankincense essential oil, geranium essential oil, ginger essential oil, grapefruit essential oil, helichrysum essential oil, hop essential oil, hyssop essential oil, juniper berry essential oil, labdanum essential oil, lemon essential oil, lemongrass essential oil, lime essential oil, magnolia essential oil, mandarin essential oil, marjoram essential oil, myrrh essential oil, myrtle essential oil, neroli essential oil, niaouli essential oil, nutmeg essential oil, orange essential oil, oregano essential oil, palmarosa essential oil, patchouli essential oil, pennyroyal essential oil, pepper black essential oil, petitgrain essential oil, pine needle essential oil, radiata essential oil, ravensara essential oil, rose essential oil, rosewood essential oil, sage essential oil, sandalwood essential oil, spearmint essential oil, spikenard essential oil, spruce essential oil, star anise essential oil, tangerine essential oil, thyme red essential oil, verbena essential oil, vetiver essential oil, wintergreen essential oil, wormwood essential oil, yarrow essential oil, or Ylang Ylang Extra essential oil, or combinations thereof.

### Hydrocarbons

In some instances, the cosmetic or personal care formulations described herein may also contain hydrocarbons, fat, oil, or wax that are not volatile (as in contrast to the essential oils, which are very volatile). In some embodiments, the hydrocarbons may include ozokerite, α-olefin oligomer, light isoparaffin, light liquid isoparaffin, squalene, squalane, vegetable squalane, ceresin, paraffin, polyethylene powder, polybutene, microcrystalline wax, liquid isoparaffin, liquid paraffin, mineral oil and vaseline.

### Fat/Wax/Non-Volatile Oil

Natural fats, wax and oils that may be suitable include but not limited to jojoba oil, carnauba wax, candelilla wax, rice wax, shellac, lanoline, mink tallow wax, spermaceti, sugar cane wax, sperm whale oil, beeswax and montan wax, argan oil, avocado oil, almond oil, olive oil, extra virgin olive oil, sesame oil, rice bran oil, rice oil, rice germ oil, corn oil, soybean oil, corn oil, persic oil, palm kernel oil, palm oil, castor oil, grape seed oil, cottonseed oil, coconut oil, hydrogenated coconut oil, beef tallow, hydrogenated oil, horse oil, mink oil, egg yolk oil, egg yolk fatty oil, rose hips oil, candlenut oil, wheat germ oil, peanut oil, camellia oil, sasanqua oil, cacao butter, Japan wax, beef bone fat, beef foot oil, hog fat, horse fat, mutton tallow, shea butter, macademia nut oil and meadowfoam seed oil.

### Viscosity Modifying Agents

The cosmetic or personal care formulations may contain one or more viscosity modifying agents, such as viscosity increasing agents. Classes of such agents include, but are not limited to, viscous liquids, such as polyethylene glycol, semisythetic polymers, such as semisynthetic cellulose derivatives, synthetic polymers, such as carbomers, poloxamers, and polyethyleneimines (e.g., PEI-10), naturally occurring polymers, such as acacia, tragacanth, alginates (e.g., sodium alginate), carrageenan, vegetable gums, such as xanthan gum, petroleum jelly, waxes, particulate associate colloids, such as bentonite, colloidal silicon dioxide, and microcrystalline cellulose, surfactants, such as PPG-2 hydroxyethyl coco/isostearamide, emulsifiers, such as disteareth-75 IPDI, and salts, such as sodium chloride, and combinations thereof.

### Antioxidants

The cosmetic or personal care formulation may contain one or more antioxidants. Examples include, but are not limited to, tocopheryls, BHT, ascorbic acid, Camellia sinensis leaf extract, ascorbyl palmitate, magnesium ascorbyl phosphate, carotenoids, resveratrol, triethyl citrate, arbutin, kojic acid, tetrahexydecyl ascorbate, superoxide dismutase, zinc, sodium metabisulfite, lycopene, ubiquinone, and combinations thereof.

### Opacifying Agents

The cosmetic or personal care formulation may contain one or more opacifying agents. Opacifying agents are added to the formulations to make it opaque. Suitable opacifying agents include, but are not limited to, glycol distearate and ethoxylated fatty alcohols.

### Ultraviolet Absorbing Agents

In some embodiments, the cosmetic formulation may include at least one ultraviolet absorbing agent. Non-limiting examples include p-aminobenzoic acid, p-aminobenzoic acid derivatives, such as ethyl p-aminobenzoate, glyceryl p-aminobenzoate, amyl p-dimethylaminobenzoate and 2-ethylhexyl p-dimethylaminobenzoate, cinnamic acid derivatives, such as benzyl cinnamate, glyceryl di-p-methoxycinnamate mono-2-ethylhexanoate, methyl 2,4-diisopropylcinnamate, ethyl 2,4-diisopropylcinnamate, potassium p-methoxycinnamate, sodium p-methoxycinnamate, isopropyl p-methoxycinnamate, 2-ethylhexyl p-methoxycinnamate, 2-ethoxyethyl p-methoxycinnamate and ethyl p-ethoxycinnamate, urocanic acid, urocanic acid derivatives, such as ethyl urocanate, benzophenone derivatives, such as 2,4-dihydroxybenzophenone, 2,2′,4,4′-tetrahydroxybenzophenone, 2-hydroxy-4-methoxy-5-sulfobenzophenone sodium, 2-hydroxy-4-methoxybenzophenone-5-sulfonic acid, 2-hydroxy-4-methoxybenzophenone, 2,2′-dihydroxy-4,4′-dimethoxybenzophenone and 2,2′-dihydroxy-4,4′-dimethoxy-5-sulfobenzophenone sodium, salicylic acid derivatives, such as ethylene glycol salicylate, 2-ethylhexyl salicylate, phenyl salicylate, benzyl salicylate, p-tert-butylphenyl salicylate, homomentyl salicylate and 3,3,5-trimethylcyclohexyl salicylate, 2-(2′-hydroxy-5′-methoxyphenyl)benzotriazole, and 4-tert-butyl-4′-methoxybenzoylmethane.

### Fragrances and Scents

In some embodiments, the cosmetic formulation may include at least one fragrance or scent. In some embodiments, the fragrance is used to mask the odor of one or more ingredients of the cosmetic formulation or the odor developed through the use of the cosmetic formulation. Suitable fragrances include, but are not limited to, vanillin. In some embodiments, the cosmetic formulation or hair treatment formulation includes about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, or 5% by weight of each or total of the fragrance, or a range defined by any two preceding values.

### Preservatives

In some embodiments, the cosmetic formulation is free or essentially free of preservatives. In some other embodiments, the cosmetic composition may include one or more preservatives, for example, gluconlactone, sodium benzoate, potassium sorbate, and combinations thereof. Other preservatives that may be used include, but are not limited to, benzoic acid, undecylenic acid, salicylic acid, sorbic acid, dehydroacetic acid, sodium dehydroacetate, isobutyl parahydroxybenzoate, isopropyl parahydroxybenzoate, ethyl parahydroxybenzoate, butyl parahydroxybenzoate, propyl parahydroxybenzoate, benzyl parahydroxybenzoate, methyl parahydroxybenzoate, sodium methyl parahydroxybenzoate, and phenoxyethanol.

Other ingredients that may be used in the cosmetic or personal care formulations described herein include, but not limited to pH adjustors, such as citric acid, sodium hydroxide, potassium hydroxide and triethanolamine.

### Conditioners

The cosmetic or personal care formulations described herein may be in the form of a conditioner. The conditioner typically includes the active agent in a suitable carrier. Additionally, the conditioner may include cationic polymers derived from polysaccharides, for example cationic cellulose derivatives, cationic starch derivatives, cationic guar derivatives and cationic locust bean gum derivatives, synthetic cationic polymers, mixtures or combinations of these agents. The formulation may include other synthetic or natural polymers or polymers derived from biological preparation processes, which are functionalized, where appropriate, for example with cationic or neutral groups. These polymers may have a stabilizing or strengthening action on the formulation, and/or a conditioning action (deposition on the surface of the skin or the hair).

The compound of Formula (I) or (II) or a combination thereof as described herein (i.e., the active agent) may be included in any suitable concentration. Typical concentrations of active agent in the conditioner range from small amounts such as at least about 0.01 wt. %, about 0.1 wt. %, and up to about 50 wt. %. Preferably the conditioner contains the active agent in a concentration ranging from 0.1 wt. % to about 10 wt. %, from about 0.5 wt. % to about 8 wt. %, or from about 1 wt. % to about 5 wt. %. While greater concentrations of active agent could be present in the conditioner, they are generally not needed to achieve the desired results. In some embodiments, the cosmetic formulation or hair treatment formulation includes, includes about, includes at most, or includes at most about, 0.1 wt. %, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. % or 20 wt. % of each or total of the conditioner, or any range of values therebetween.

### Shampoos

The cosmetic or personal care formulations described herein may be in the form of a shampoo. The shampoo typically includes the compound of Formula (I) or (II) or a combination thereof as described herein (i.e., the active agent) in a suitable carrier. The active agent may be included in any suitable concentration. Typical concentrations of active agent in the shampoo range from small amounts such as at least about 0.01 wt. %, about 0.1 wt. %, and up to about 50 wt. %. Preferably the conditioner contains the active agent in a concentration ranging from 0.1 wt. % to about 10 wt. %, from about 0.5 wt. % to about 8 wt. %, or from about 1 wt. % to about 5 wt. %. While greater concentrations of active agent could be present in the shampoo, they are generally not needed to achieve the desired results. Additionally, the shampoo may include from about 0.5 wt. % to about 20 wt. % of one or more surfactants, and/or one or more cosmetically acceptable excipients described herein. In some embodiments, the cosmetic formulation or hair treatment formulation includes, includes about, includes at most, or includes at most about, 0.1 wt. %, 0.5 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 15 wt. %, 20 wt. %, 25 wt. %, 30 wt. %, 35 wt. %, 40 wt. %, 45 wt. %, 50 wt. % of each or total of the shampoo, or any range of values therebetween.

### Creams, Lotions, Gels, and Polish

The cosmetic or personal care formulations described herein may be in the form of a cream, lotion, gel, or polish. The cream, lotion, gel, or polish typically includes the compound of Formula (I) or (II) or a combination thereof as described herein (i.e., the active agent) in a suitable carrier. The active agent may be included in any suitable concentration. Typical concentrations of active agent in the shampoo range from small amounts such as at least about 0.01 wt. %, about 0.1 wt. %, and up to about 50 wt. %. Preferably the cream, lotion, gel, or polish contains the active agent in a concentration ranging from 0.1 wt. % to about 10 wt. %, from about 0.5 wt. % to about 8 wt. %, or from about 1 wt. % to about 5 wt. %. While greater concentrations of active agent could be present in the cream or lotion, they are generally not needed to achieve the desired results. Additionally, the cream, lotion, gel or polish may include one or more cosmetically acceptable excipients described herein.

### Sprays

The cosmetic or personal care formulations described herein may be in the form of a spray. The spray typically includes the compound of Formula (I) or (II) or a combination thereof as described herein (i.e., the active agent) and a cosmetically acceptable carrier. Preferably, the active agent in a concentration ranging from 0.1 wt. % to about 10 wt. %, from about 0.5 wt. % to about 8 wt. %, or from about 1 wt. % to about 5 wt. %. In some embodiments, the carrier is water or a water and alcohol (e.g., ethanol) mixture. The spray formulation optionally includes an antioxidant, sunscreen agent, vitamin, protein, peptide, plant extract, humectant, oil, emollient, lubricant, thickener, hair conditioning agent, polymer, and/or surfactant. In some embodiments, the spray formulation includes a preservative. In some embodiments, the cosmetic or personal care formulation includes a fragrance. In some embodiments, the formulation includes a surfactant. In some embodiments, the formulation contains water, fragrance, a preservative, and an active agent. In some embodiments, the formulation contains water, fragrance, a preservative, and an active agent. In some embodiments, the formulation contains water, a preservative, fragrance, an active agent, and an anti-static agent. In some embodiments, the formulation contains water, a preservative, fragrance, an active agent, and a hair conditioning agent. In some embodiments, the formulation contains water, a preservative, fragrance, an active agent, and a surfactant.

The spray formulations may be dispensed from containers that include aerosol dispensers or pump spray dispensers. Such dispensers are known in the art and are commercially available from a variety of manufacturers.

### Propellants

When the spray formulation is dispensed from a pressurized aerosol container, a propellant may be used to disperse the formulation out of the container. Suitable propellants include, but are not limited to, a liquefiable gas or a halogenated propellant. Examples of suitable propellants include dimethyl ether and hydrocarbon propellants such as propane, n-butane, isobutane, CFCs, and CFC-replacement propellants. The propellants may be used singly or admixed.

The amount of propellant may range from about 10% to about 60% by weight of the formulation. The propellant may be separated from the hair repair formulation as in a two-compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the formulation to the hair.

Additionally, the formulation, depending on use, may include an oil, a hair conditioning agent, and/or a thickening agent. The cream, lotion, gel, or polish may also include a fragrance, a plant extract, and/or a surfactant. The cream, lotion, gel, or polish may be packaged in a tube, tub, bottle (e.g., narrow tipped applicator bottle), or other suitable container.

### Kits

Some embodiments described herein relate to a kit that can include a cosmetic formulation including a compound of Formula (I) or Formula (II), or a combination thereof. In some embodiments, the cosmetic formulation includes or is a hair treatment formulation. In further embodiments, the cosmetic formulation is a hair treatment formulation, for example, a hair styling formulation (e.g., a hair relaxing formulation, a hair curling formulation, a hair perming formulation and/or a hair straightening formulation). In some embodiments, a hair treatment formulation is combined with additional ingredients or formulation components to form the cosmetic formulation.

In some embodiments, the kit described herein is a hair care kit. In some further embodiments, the kit is a hair styling kit (e.g., a hair relaxing kit, a hair curling kit, a hair straightening kit, a hair perming kit). In some embodiments of the kit described herein, the kit may further include one or more hair treatment agents. In some embodiments, the one or more hair treatment agents are separated from the formulation containing the compound of Formula (I) or (II) as described herein. For example, the one or more hair treatment agents are in a different container or compartment from the compound of Formula (I) or (II). In some embodiments, different containers may be different bottles (e.g., narrow tipped applicator bottle), pods, pouches, sachets, or capsules, or combinations thereof. In some embodiments, different compartments may be different compartments of the same bottle, pod, pouch, sachet, capsule, or combinations thereof. For example, in some embodiment, a pod contains a styling agent (e.g. a reducing agent, such as a sulfite salt) and a different pod contains the compound of Formula (I) or (II), or the styling agent (e.g. reducing agent, such as a sulfite salt) and the compound of Formula (I) or (II) are contained within the same pod and housed in different compartments. In some embodiments, the structure of the pod is formed from a hydrolyzed poly(vinyl alcohol). In some embodiments, a pod may be multilayered (e.g. include a plurality of poly(vinyl alcohol) layers). In some embodiments, the one or more hair treatment agents includes a styling agent (e.g., a relaxing agent, a reducing agent, or combinations thereof. In some embodiments, the relaxing agent is selected from sodium hydroxide, lithium hydroxide, potassium hydroxide, guanidinium hydroxide, ammonium hydroxide, or combinations thereof. In some embodiments, a reducing agent is selected from a sulfite salt, a thioglycolate salt, thioglycolic acid, thiolactic acid, dihydrolipoate, thioglycerol, mercaptopropionic acid, sodium bisulfite, ammonium bisulfide, zinc formaldehyde sulfoxylate, sodium formaldehyde sulfoxylate, sodium metabisulfite, potassium borohydride, pegylated thiols and hydroquinone, and combinations thereof. In some embodiments, the sulfite salt or sulfite salt reducing agent is sodium sulfite or potassium sulfite, or a combination thereof.

In another embodiment of the kit described herein, the kit is a hair relaxing kit. The kit may include separate containers or compartments for one or more relaxing agents e.g., a sulfite salt), and the compound of Formula (I) or (II) as described herein (such as a composition including the compound of Formula (I) or (II) and one or more surfactants or other cosmetically acceptable excipients or carrier).

In another embodiment of the kit described herein, the kit is a hair styling kit (e.g., a hair curling kit, a hair relaxing kit, a hair perming kit, a hair straightening kit). The kit may include separate containers or compartments. One container or compartment includes the active agent (such as a composition including the compound of Formula (I) or (II) and one or more surfactants or other cosmetically acceptable excipients or carrier). A second container for mixing the active agent with a cosmetically acceptable carrier (such as purified water). In some embodiments, I kit may include separate containers or compartments for one or more reducing agents, and the active agent (such as a composition including the compound of Formula (I) or (II) and one or more surfactants or other cosmetically acceptable excipients or carrier). In some embodiments, the kit does not include an oxidizing agent (such as a peroxide or hydrogen peroxide).

In some embodiments of the kit described herein, an active agent formulation containing a compound of Formula (I) or (II) or a combination thereof (i.e., the active agent) is provided, which is mixed at the time of use with a second formulation, such as a styling (e.g., relaxing, straightening, curling, and/or perming) formulation, or a combination thereof. In these embodiments, the active agent formulation may contain any suitable concentration of active agent in a suitable carrier, typically a diluent, such as described above. The concentration of the active agent is suitable to provide a mixture with the appropriate final volume and final concentration of the active agent. In some embodiments, an active agent formulation can contain a concentration of active agent at, at about, at least, or at least about, 0.1 wt. %, 0.5 wt. %, 0.8 wt. %, 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, 10 wt. %, 11 wt. %, 12 wt. %, 13 wt. %, 14 wt. %, 15 wt. %, 20 wt. %, 25 wt. %, 30 wt. %, 35 wt. %, 40 wt. %, 45 wt. %, 50 wt. %, 60 wt. %, 70 wt. % or 80 wt. %, or a range defined by any two preceding values. For example, an active agent formulation can contain a concentration of active agent ranging from about 1 wt. % to about 50 wt. %, from about 2 wt. % to about 25 wt. %, or from about 5 wt. % to about 10 wt. %.

In particular embodiments of the hair treatment kit, the kit typically contains a first formulation for styling (e.g., straightening, curing, relaxing and/or perming) hair. The first formulation may be formulated as two or more components which may be mixed together before application to the hair. For example, the first formulation may be in the form of two components, such as a styling agent (e.g., a relaxing agent or a reducing agent, such as a sulfite salt) and the active agent. Suitable relaxing agents may be selected from sodium hydroxide, lithium hydroxide, potassium hydroxide, guanidinium hydroxide, ammonium hydroxide, or combinations thereof. Suitable reducing agents may be selected from a sulfite salt, or a thioglycolate salt, thioglycolic acid, thiolactic acid, dihydrolipoate, thioglycerol, mercaptopropionic acid, sodium bisulfite, ammonium bisulfide, zinc formaldehyde sulfoxylate, sodium formaldehyde sulfoxylate, sodium metabisulfite, potassium borohydride, pegylated thiols and hydroquinone. The components of the first formulation may differ depending on the hair coloring treatment desired (such as for semi-permanent, demi-permanent, or permanent hair color), the texture of the hair, the sensitivity of the user's skin, and such the like. The kit also includes a second formulation containing an effective amount of the compound of Formula (I) or (II) or a combination thereof (i.e., the active agent formulation). Suitable formulations containing the surface agents are discussed above. The second formulation may be in any suitable form. Suitable forms include, but are not limited to, non-viscous liquids, low to moderate viscosity liquids, pastes, lotions, milks, mousses, sprays, gels, creams, shampoos, conditioners, and the like. In further embodiments, the active agent formulation includes, comprises, consisting of, or consisting essentially of the compound of Formula (I) or (II), and one or more surfactants. The second formulation will be present in a suitable container or compartment, which depends on the form of the formulation. In some embodiments, the active agent formulation is provided as two or more separate ingredients. For example, the active agent may be provided in a sealed package and the excipient provided in a vial, another container, or another compartment. A suitable mixing container for the active agent and the excipient may be provided. In some embodiments, the active agent formulation (or second formulation) is mixed with the first formulation (e.g., hair styling treatment formulation), and the mixture is applied to the hair.

In some embodiments, a formulation component (e.g., active agent, hair treatment agent) and/or a formulation (e.g., first formulation, second formulation, active agent formulation, hair treatment formulation) are in the form of a pill (e.g., a tablet or capsule). In some embodiments, the pill is configured to be combined with (e.g., dissolved within) another cosmetic formulation component (e.g., a cosmetically acceptable carrier, such as water) prior to application to the hair. In some embodiments, the pill may be combined with another cosmetic formulation components for, for about, for at least, for at least about, for at most, or for at most about, 1 min, 2 min, 5 min, 10 min, 15 min, 20 min, 25 min, 30 min, 45 min, 1 hour, 2 hours, 6 hours, 12 hours, 18 hours, 1 day, 2 days, 3 days, 1 week, 2 weeks or 1 month before application to the hair. In some embodiments, the pill is provided in a tube, a blister pack, a sachet, and/or a bottle.

In some embodiments, the kit further includes an applicator bottle (e.g., narrow tipped applicator bottle), spray bottle, gloves, shampoo, conditioner, and/or an odor eliminator. Instructions for use of the kit are also typically provided. In some embodiments, the kit contains more than one container (or more than one compartment in a given container). In some embodiments, the kit further includes a styling tool (e.g., a heated styling tool and/or a mechanical styling stool (e.g., hair rollers, clips, combs and/or brushes)).

### Other Materials in the Cosmetic Formulation or Kit

The kit for hair treatment may optionally contains shampoos and/or conditioners. The kit may further contain an odor eliminator. The odor eliminator can be incorporated into the first or second formulation, or a mixture thereof. Alternately, the odor eliminator is present in a suitable container or compartment for use before or after washing the second formulation from the hair. Some suitable odor eliminators are known to those of ordinary skill in the art.

In some embodiments of the cosmetic formulation or kit described herein, the cosmetic formulation or kit may further include small or trace amount of maleic acid or a maleate salt (i.e., maleic acid is anionic form with a cationic counterion). In some embodiments, the maleic acid or salt thereof in the cosmetic formulation, hair treatment formulation or kit is, is about, or less than about 0.5 wt. %, 0.4 wt. %, 0.3 wt. %, 0.2 wt. %, 0.1 wt. %, 0.09 wt. %, 0.08 wt. %, 0.07 wt. %, 0.06 wt. %, 0.05 wt. %, 0.04 wt. %, 0.03 wt. %, 0.02 wt. %, or 0.01 wt. %, or in a range defined by any two of the preceding values.

### Methods of Use

Some embodiments provided herein relate to a method of treating hair that can include administering to a subject a cosmetic formulation that can include an effective amount of one or more diester compounds described herein, for example a compound of Formula (I) or Formula (II). Some embodiments provided herein relate to a method of treating hair that can include administering and/or applying to hair a cosmetic formulation that can include an effective amount of one or more diester compounds of Formula (I) or Formula (II) or a combination thereof, as described herein. In some embodiments, administering and/or applying the cosmetic formulation may be performed by spraying, rubbing, pouring and/or brushing the cosmetic formulation onto the hair, and/or dipping the hair into the cosmetic formulation. For example, in some embodiments, the cosmetic formulation can be applied to the hair from a narrow tipped applicator bottle allowing controlled application of the cosmetic formulation. In some embodiments, the cosmetic formulation (e.g. hair treatment formulation or hair styling formulation) is formed by mixing the one or more diester compounds described herein (e.g., a compound of Formula (I) or Formula (II)) with a cosmetically acceptable carrier and/or hair treatment agent.

In some embodiments, the compound of Formula (I) or (II) is in a separate composition (i.e., active agent formulation). The active agent formulation may include at least or up to 5%, 10%, 15% or 20% of one or more surfactants (for example, a cationic surfactant such as behentrimonium chloride and/or behentrimonium methosulfate, and/or an anionic sufactant) prior to combining with the hair treatment formulation. As such, the cosmetic formulation is formed by combining the hair treatment formulation with the composition including the compound of Formula (I) or (II).

In some embodiments, the method further includes rinsing the hair after applying the cosmetic formulation. In further embodiments, the method may further include applying a hair conditioning formulation (e.g., cream, gel, paste, spray, etc.) to the hair after the rinsing step. In some such embodiments, the hair conditioning formulation may include the compound of Formula (I) or (II) described herein, for example, the hair treatment formulation described herein used in the method of treating hair to reduce or prevent hair breakage. In some further embodiments, the breakage of the hair is decreased by at least 5%, 10%, 15% or 20%, compared to hair treated with the cosmetic formulation in the absence of the compound of Formula (I) or (II). For example, hair breakage may be decreased by at least 10%, 15%, 20%, 25%, 30%, 35%, 40%, 45%, 50% compared to untreated hair, when the cosmetic formulation or hair styling formulation is used at least 2-3 times/week for at least 2 weeks, 3 weeks, 1, 2, 3, 4, 5 or 6 months.

Some additional embodiments relate to a method of treating hair to reduce or prevent hair breakage, including: applying a hair treatment formulation to damaged hair, wherein the hair treatment formulation includes a compound of Formula (I) or (II) or a combination thereof, and a cosmetically acceptable carrier, wherein breakage of the hair is decreased by at least 5% compared to untreated hair, or hair treated with a repair formulation that does not contain the active agent described herein (i.e., the diester compound). In some embodiments, the amount of compound of Formula (I) or (II) in the hair treatment formulation is at least about 1%, 2%, 3%, 4%, 5%, 6%, 7%, 8%, 9%, 10%, 15% or 20% by weight, or a range defined by any two of the preceding values. For example, the amount of compound of Formula (I) or (II) in the hair treatment formulation is at least about 5% to about 10% when the hair is coarse or heavily damaged (e.g., have been previously subject to repeated coloring, bleaching, perming, relaxing, straightening, or heat styling, etc.). In some embodiments, the damaged hair is caused by bleaching, relaxing, coloring, perming, straightening, or heat styling, or combination thereof. In some embodiments, the hair treatment formulation is applied to the hair 1, 2, 3, 4, 5, 6, 7, 8, 9 or 10 times a week, to achieve the decreased hair breakage and improved hair quality over time. For example, hair breakage may be decreased by at least 10%, 15%, 20%, 25%, 30%, 35%, 40%, 45%, 50% compared to untreated hair, when the cosmetic formulation or hair styling formulation is used at least 2-3 times/week for at least 2 weeks, 3 weeks, 1, 2, 3, 4, 5 or 6 months.

In some embodiments, the hair treatment formulation is formed by mixing the compound of Formula (I) or (II) in a cosmetically acceptable carrier. In some such embodiments, the cosmetically acceptable carrier includes or is water, purified water, or deionized water. In some embodiments, the compound of Formula (I) or (II) is in a separate composition including at least or at most about 5% to 20% of one or more surfactants (for example, a cationic surfactant such as behentrimonium chloride and/or behentrimonium methosulfate) prior to mixing with the cosmetically acceptable carrier. As such, the hair treatment formulation is formed by combining the composition including the compound of Formula (I) or (II) with the cosmetically acceptable carrier (e.g., purified water). In some embodiments, the method further includes rinsing the hair after applying the hair treatment formulation. In other embodiments, the method does not require rinsing the hair after applying the hair treatment formulation. In further embodiments, applying the hair treatment formulation includes spraying the hair treatment formulation onto the damage hair. In further embodiments, applying the hair treatment formulation includes pouring the hair treatment formulation onto hair from a narrow tipped applicator bottle.

Some embodiments relate to a method of styling (e.g., curling, straightening, relaxing, perming) hair, including: applying a cosmetic formulation or hair styling formulation to the hair, wherein the cosmetic formulation or hair styling formulation includes a compound of Formula (I) or (II) or a combination thereof as described herein; and styling the hair (e.g., curling, straightening, relaxing, perming, rolling, brushing, combing). In some embodiments, the cosmetic formulation styles or aids in styling hair by changing the texture of hair (e.g., smoothing the hair texture, decreasing (i.e., managing) hair frizz, increasing combability of the hair, improving touch (e.g., decreasing coarse feel of hair) and/or increasing shine of the hair). In some embodiments, the cosmetic formulation strengthens or aids in strengthening hair, for example the cosmetic formulation may strengthen or aid in strengthening hair relative to untreated hair and/or to a control formulation applied to hair (e.g., a control formulation without a compound of Formula (I) and/or (II)). In some embodiments, styling the hair may include treating hair with a styling tool (e.g., heated hair styling tool and/or mechanical styling tool (e.g., hair roller, comb, brush and/or weight)). For example, in some embodiments, hair rollers may be utilized to curl hair, and brushing and/or combing may be used to straighten hair. In some embodiments, the method further includes drying (e.g., blow drying) the hair prior and/or subsequently to treating the hair with the styling tool. In some embodiments, the cosmetic formulation or hair styling formulation is applied to the hair (with or without styling the hair) 1, 2, 3, 4, 5, 6, 7, 8, 9 or 10 times a week, or 1, 2, 3, 4, 5, 6, 7, 8, 9 or 10 times a month, to achieve a desired hair texture or style. In some embodiments, the application of the method of styling using the cosmetic formulation or hair styling formulation retains, substantially retains, or partially retains the hair style and/or texture even after 1, 2, 3, 4, 5, 6, 7, 8, 9, 10, 11, 12, 13, 14, 15, 16, 17, 18, 19, 20, 25, 30, 35, 40, 45 or 50 wash cycles (e.g., rinsing, shampooing and/or conditioning) of the hair. In some embodiments, the application of the method of styling using the cosmetic formulation or hair styling formulation retains, substantially retains, or partially retains the hair style and/or texture even after 1, 2, 3, 4, 5, 6, 7, 8, 9, 10, 11, 12, 13, 14, 15, 16, 17, 18, 19 or 20 days after treatment. For example, in some embodiments, repeated application of the method of styling using the cosmetic formulation or hair styling formulation increases or aids in increasing the retention of the hair style and/or texture.

In further embodiments, the amount of compound of Formula (I) or (II) in the cosmetic formulation or hair styling formulation is, is about, is at least, or is at least about 0.5%, 1%, 2%, 3%, 4%, 5%, 6%, 7%, 8%, 9%, 10%, 12%, 15%, 18%, 20%, 25%, 30%, 35%, 40%, 45% or 50% by weight, or any range of values therebetween. For example, the amount of compound of Formula (I) or (II) in the cosmetic formulation or hair styling formulation is at least about 5% to about 10% when the hair is coarse or heavily damaged (e.g., have been previously subject to repeated coloring, bleaching, perming, relaxing, straightening, or heat styling, etc.). In some embodiments, the method further includes rinsing, shampooing and/or conditioning the hair after applying the cosmetic formulation or hair styling formulation. In other embodiments, the method does not require or include rinsing, shampooing and/or conditioning the hair after applying the cosmetic formulation or hair styling formulation. In some embodiments, the styling method changes the texture of the hair. In some embodiments, the method relaxes, straightens, curls or perms the hair. In some embodiments, the method styles the hair permanently or semi-permanently. In some embodiments, the cosmetic formulation or hair styling formulation is applied to the hair 1, 2, 3, 4, 5, 6, 7, 8, 9 or 10 times a week (e.g., to achieve the style, texture, decreased hair breakage and/or improved hair quality over time). For example, hair breakage may be decreased by at least 10%, 15%, 20%, 25%, 30%, 35%, 40%, 45%, 50% compared to untreated hair, when the cosmetic formulation or hair styling formulation is used at least 2-3 times/week for at least 2 weeks, 3 weeks, 1, 2, 3, 4, 5 or 6 months.

Some embodiments relate to a method of perming hair, including: applying a reducing agent to the hair; and applying a cosmetic formulation or perming formulation to the hair, wherein the cosmetic formulation or perming formulation includes a compound of Formula (I) or (II) or a combination thereof as described herein. Some additional embodiments relate to a method of perming hair, including: applying a applying a cosmetic formulation or perming formulation to the hair, wherein the cosmetic formulation or perming formulation includes a reducing agent and a compound of Formula (I) or (II) or a combination thereof as described herein. In further embodiments, the amount of compound of Formula (I) or (II) in the cosmetic formulation or perming formulation is, is about, is at least, or is at least about 0.5%, 1%, 2%, 3%, 4%, 5%, 6%, 7%, 8%, 9%, 10%, 12%, 15%, 18%, 20%, 25%, 30%, 35%, 40%, 45% or 50% by weight, or any range of values therebetween. For example, the amount of compound of Formula (I) or (II) in the cosmetic formulation or perming formulation is, is about, is at least, or is at least about 5-10 wt. % or 0.1-20 wt. % when the hair is coarse or heavily damaged (e.g., have been previously subject to repeated coloring, bleaching, perming, relaxing, straightening, or heat styling, etc.). In some embodiments, the method further includes rinsing and/or washing the hair after applying the reducing agent. In some embodiments, the method further includes rinsing and/or washing the hair after applying the cosmetic formulation or perming formulation. In further embodiments, the method may also include styling hair with a hair styling tool. In some embodiments, the reducing agent is selected from a sulfite salt, a thioglycolate salt, thioglycolic acid, thiolactic acid, dihydrolipoate, thioglycerol, mercaptopropionic acid, sodium bisulfite, ammonium bisulfide, zinc formaldehyde sulfoxylate, sodium formaldehyde sulfoxylate, sodium metabisulfite, potassium borohydride, pegylated thiols, or hydroquinone, or combinations thereof. In some embodiments, the reducing agent is applied to the hair prior to the cosmetic formulation or perming formulation being applied to the hair. In some such embodiments, the cosmetic formulation or perming formulation is applied to the hair at, at about, at most, at most about, at least, or at least about, 30 seconds, 1 minute, 5 minutes, 10 minutes, 15 minutes, 30 minutes, or 1 hour after the application of the reducing agent. In some embodiments, the reducing agent is applied to the hair concurrently with the styling formulation. In some embodiments, the styling formulation include the reducing agent. In some embodiments, the styling formulation does not include an oxidizing agent. In some embodiments, the method achieves styling of the hair in a single step. In some embodiments, the method does not include applying an oxidizing agent to the hair subsequent to applying the styling formulation and/or treating the hair with a styling tool. In some embodiments, the method including applying the cosmetic formulation or perming formulation with the active agent (e.g., the compound of Formula (I) or (II)) may achieve decreased hair breakage and improved hair quality compared to a perming method without the active agent. For example, hair breakage may be decreased by at least 10%, 15%, 20%, 25%, 30%, 35%, 40%, 45%, 50% compared to using a cosmetic formulation or perming formulation without the active agent.

In some embodiments, the method further includes washing, rinsing, or drying, or a combination thereof, of the hair after the application of the cosmetic formulation. In some embodiments, the method of styling hair (e.g., perming hair) may enable the hair to be washed subsequent to subsequent to applying the styling formulation and/or treating the hair with a styling tool while maintaining or substantially maintaining the styling of the hair achieved by the method. In some embodiments, the hair may be washed prior to or without the application of an oxidizing agent to the hair while maintaining or substantially maintaining the styling of the hair achieved by the method. In some embodiments, the number of times the hair is washed without loss of the hair style achieved by the method is, is about, is at least, or is at least about, 1, 2, 3, 4, 5, 6, 7, 8, 9, 10, 12, 15, 20, 25, 30, 35, 40, 45 or 50 times, or any range of values therebetween.

In some embodiments, a hair styling tool is applied to the hair prior to, subsequent to and/or concurrently with the application of the cosmetic formulation. For example, in some embodiments, the hair is put into hair rollers prior to application of the cosmetic formulation. In a further example, in some embodiments, the cosmetic formulation is applied to the hair and the hair is subsequently rolled into hair rollers. In an additional example, in some embodiments, the hair is mechanically straightened (e.g. by combing, brushing, weights) subsequent to the application of the cosmetic formulation to the hair (e.g., with or without use of a hair styling tool). In a further additional example, in some embodiments, the hair is mechanically straightened prior to the application of the cosmetic formulation to the hair (e.g., with or without use of a hair styling tool). In some embodiments, the hair is rinsed prior to, subsequent to and/or concurrently with the removal of the styling tool. For example, in some embodiments, the hair is rinsed prior to removing the hair rollers. In a further example, in some embodiments, the hair is removed from hair rollers prior to being rinsed.

### Methods of Using Hair Treatment Kits

In some embodiments, the active agent formulation is in a kit and the kit may further include additional hair treatment agent(s) or formulation(s), such as a hair styling (e.g., a hair relaxing, a hair perming, a hair curling, a hair straightening) agent or formulation. In some embodiments, the active agent formulation containing the diester compound(s) is applied to the hair after the application of the hair treatment agent(s). In some embodiments, the active agent formulation is applied to the hair at, at about, at most, at most about, at least, or at least about, 30 seconds, 1 minute, 5 minutes, 10 minutes, 15 minutes, 30 minutes, 1 hours, 2 hours, 3 hours, 4 hours, 6 hours, 12 hours, 18 hours, 24 hours or 30 hours after the application of the hair treatment agent(s). In some embodiments, the active agent formulation is applied to the hair at least 24 hours after the application of the hair treatment agent(s). In other embodiments, the active agent formulation is applied to the hair within about 1 to 3 hours, within about 30 minutes, or within about 1 minute after the application of the hair treatment agent(s). In some embodiments, the hair is washed, rinsed, and/or dried to substantially remove any remaining hair treatment agent prior to the application of the active agent formulation.

In some other embodiments, the active agent formulation is applied to the hair prior to the application of the hair treatment agent(s). In some embodiments, the cosmetic formulation is applied to the hair at, at about, at least, or at least about, 30 seconds, 1 minute, 5 minutes, 10 minutes, 15 minutes, 30 minutes, 1 hours, 2 hours, 3 hours, 4 hours, 6 hours, 12 hours, 18 hours, 24 hours or 30 hours prior the application of the hair treatment agent(s). In some embodiments, the active agent formulation is applied to the hair at least 24 hours prior to the application of the hair treatment agent(s). In other embodiments, the active agent formulation is applied to the hair within about 1 to 3 hours, within about 30 minutes, or within about 1 minute prior to the application of the hair treatment agent(s). In some embodiments, the hair is washed, rinsed, and/or dried to substantially remove any remaining cosmetic formulation prior to the application of the hair treatment agent(s).

In some embodiments, the active agent formulation and the hair treatment agent(s) are applied to the hair simultaneously. In some embodiments, the active agent formulation and the hair treatment agent(s) are premixed as a hair application mixture prior to being applied to the hair. In some embodiments, the hair application mixture is premixed at, at about, at most, at most about, at least, or at least about, 30 seconds, 1 minute, 5 minutes, 10 minutes, 15 minutes, 30 minutes, 1 hours, 2 hours, 3 hours, 4 hours, 6 hours, 12 hours, 18 hours, 24 hours or 30 hours before being applied to the hair. In some embodiments, the hair application mixture is premixed at least 24 hours prior to being applied to the hair. In other embodiments, the hair application mixture is premixed within about 1 to 3 hours, within about 30 minutes, or within about 1 minute before being applied to hair. In some embodiments, the method further includes rinsing the hair after the application of the hair application mixture.

Hair breakage is a significant problem encountered during bleaching, relaxing, straightening, curling, perming, coloring and other treatments of hair. Applying the cosmetic formulation described herein to hair may achieve a reduced breakage of the hair relative to hair treated with a hair treatment agent (e.g. a styling formulation) and/or cosmetic formulation in the absence of the compound of Formula (I) or Formula (II). In some embodiments, breakage of the hair is decreased by, by about, by at least, or by at least about, 0.5%, 1%, 2%, 3%, 4%, 5%, 6%, 8%, 10%, 12%, 15%, 20%, 25%, 30%, 35%, 40%, 45% or 50%, or a range defined by any two of the preceding values. In some embodiments, the cosmetic formulation is applied to untreated hair. In some embodiments, the untreated hair is virgin hair, or is bleached hair and/or colored hair, or is damaged hair has been treated with other hair repair agent that does not contain the diester compound described herein. In some embodiments, the hair breakage is reduced by at least 10%, 15%, 20%, 25%, 30%, 35%, 40%, 45%, or 50%, compared to untreated hair, when the cosmetic formulation containing compound of Formula (I) or Formula (II) have been used at least 1-3 times/week for at least 2 weeks, 3 week, 1, 2, 3, 4, 5 or 6 months.

In any embodiments of the method described herein, the cosmetic formulation or the active agent formulation includes a compound of Formula (I), where each of R¹ and R² is independently selected from the group consisting of an optionally substituted C₈ alkyl, and each of R^{A} and R^{B} is hydrogen. In further embodiments, the compound of Formula (I) is dioctyl maleate or bis(2-ethylhexyl)maleate, or a combination thereof.

### EXAMPLES

Additional embodiments are disclosed in further detail in the following examples, which are not in any way intended to limit the scope of the claims.

### Example 1: Straightening Mannequin Hair

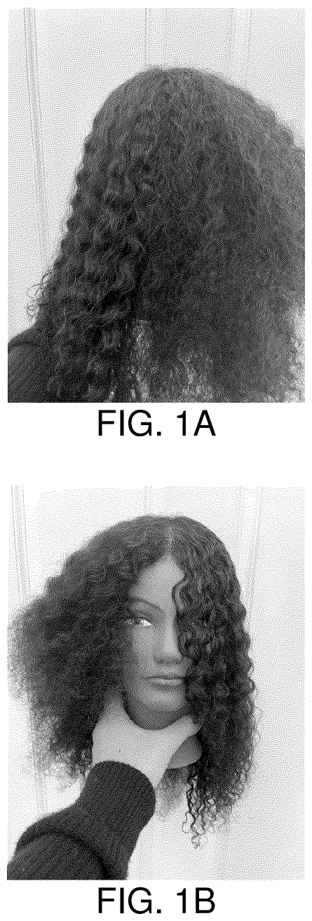

The left side of a mannequin's hair with curly human hair (Janet model mannequin head from Pivot Point) was straightened using Cosmetic Formulation 1 shown in Table 1. Cosmetic Formulation 1 was applied to the left side of the hair via a spray bottle until soaking wet. The left side of the hair was combed for 35 min, and then rinsed for 3 min in lukewarm water and air dried. After 24 hours both sides of the hair were shampooed, conditioned and air dried. FIGS. 1A and 1B show the final results of the treated left side of the mannequin's hair (i.e., the mannequin's left) and the control right side of the mannequin's hair (i.e., the mannequin's right). FIGS. 1A and 1B demonstrate that Cosmetic Formulation 1 is able to straighten curly hair, and that the combability, shine, and health of the hair was noticeably improved on the treated side.

TABLE 1
Cosmetic Formulation 1

| Compounds | Amount (wt. %) |
|---|---|
| water | 86.7 |
| bis(2-ethylhexyl) maleate | 8.44 |
| ethanol | 0.49 |
| oleyl alcohol | 0.49 |
| stearamidopropyl dimethyl amine | 0.33 |
| sodium sulfite | 3.09 |
| sodium cocoyl isethionate | 0.22 |
| vanillin | 0.24 |

### Example 2: Curling Mannequin Hair

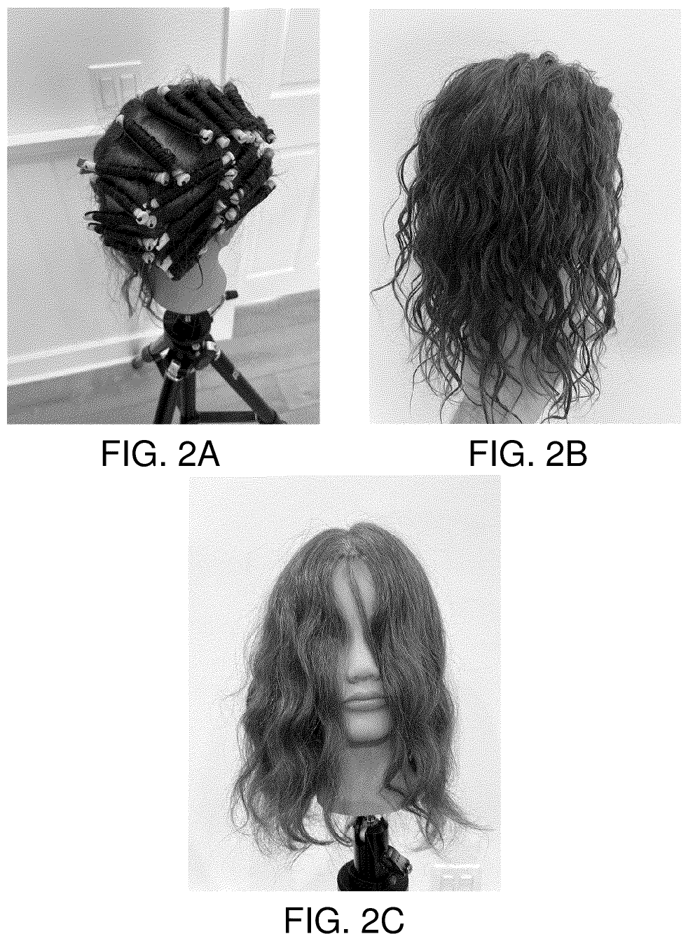

The right side of a mannequin's hair with straight human hair (Erica model mannequin head from Pivot Point) was rolled wet with water using yellow perm rods (i.e., 0.28 inch perm rods), and the resultant hair is shown in FIG. 2A. Cosmetic Formulation 2 shown in Table 2 was added via applicator bottle to the right side (i.e. the mannequin's right) to fully soak the hair to a dripping wet amount. The applied Cosmetic Formulation 2 was left to sit on the hair for 30 min. The perm rods were removed from the right side, and the right side of the hair was rinsed under lukewarm water for 3 min and left to air dry, and the resultant hair is shown in FIG. 2B. Both sides of the hair were washed with shampoo and conditioner after 24 hours, and was washed and dried another 10 times over 10 days, and the resultant hair is shown in FIG. 2C. FIG. 2C shows that the curl pattern of the right side of the mannequin's hair treated with Cosmetic Formulation 2 was significantly retained over the ten washes relative to the untreated left control side of the hair, and the treated right side of the hair showed improved health compared to untreated right side.

TABLE 2
Cosmetic Formulation 2

| Compounds | Amount (wt. %) |
|---|---|
| water | 88.05 |
| bis(2-ethylhexyl) maleate | 7.61 |
| ethanol | 0.44 |
| oleyl alcohol | 0.44 |
| stearamidopropyl dimethyl amine | 0.3 |
| sodium sulfite | 2.94 |
| sodium cocoyl isethionate | 0.22 |

### Example 3: Curling Bleached Swatches

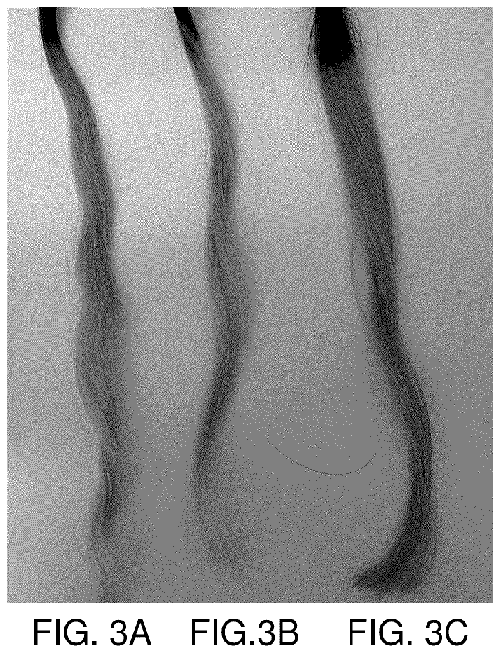

Previously bleached hair swatches from the same head were obtained. The hair swatches were rolled wet with water, with one hair swatch rolled in using a yellow perm rod (i.e., 0.28 inch perm rods) and another hair swatch rolled using an orange perm rod (i.e., 0.87 inch perm rod). A control hair swatch was not rolled. Cosmetic Formulation 2 shown in Table 2 was applied via an applicator bottle to the rolled hair swatches for 30 min to saturate. The rolled and treated swatches were rinsed for one min, removed from the rods, and let air dry. After 24 hours, the rolled and treated and the control hair swatches were shampooed and conditioned. The swatches were further shampooed, conditioned and air dried 10 times. FIG. 3A shows the resultant treated hair swatch curled with a yellow perm rod, FIG. 3B shows the resultant treated hair swatch curled with an orange perm rod, and FIG. 3C shows the untreated hair swatch from the same head that was not curled. FIGS. 3A and 3B demonstrate that bleached hair is able to be permed using Cosmetic Formulation 2 with substantial curl retention through 10 washes, and that Cosmetic Formulation 2 provides excellent hair condition and shine.

### Example 4: Additional Formulations

Additional example Cosmetic Formulations 3-5 that were prepared are shown in Tables 3-5 below.

TABLE 3
Cosmetic Formulation 3

| Compounds | Amount (wt. %) |
|---|---|
| water | 88.1 |
| bis(2-ethylhexyl) maleate | 5.58 |
| ethanol | 0.32 |
| oleyl alcohol | 0.32 |
| stearamidopropyl dimethyl amine | 0.23 |
| sodium sulfite | 4.84 |
| sodium cocoyl isethionate | 0.21 |
| vanillin | 0.4 |

TABLE 4
Cosmetic Formulation 4

| Compounds | Amount (wt. %) |
|---|---|
| water | 88.99 |
| bis(2-ethylhexyl) maleate | 8.19 |
| ethanol | 0.47 |
| oleyl alcohol | 0.47 |
| stearamidopropyl dimethyl amine | 0.33 |
| sodium sulfite | 1.4 |
| sodium cocoyl isethionate | 0.15 |

TABLE 5
Cosmetic Formulation 5

| Compounds | Amount (wt. %) |
|---|---|
| water | 89.23 |
| bis(2-ethylhexyl) maleate | 8.08 |
| ethanol | 0.47 |
| oleyl alcohol | 0.47 |
| stearamidopropyl dimethyl amine | 0.32 |
| sodium sulfite | 1.25 |
| sodium cocoyl isethionate | 0.18 |

### Example 5: Repeated Straightening Mannequin Hair

The left side of three mannequins' hair with curly human hair (Janet model mannequin head from Pivot Point) was straightened using Cosmetic Formulation 6 shown in Table 6, with some of mannequins' hair repeatedly treated. The ingredients of Cosmetic Formula 6 were mixed and applied fresh at room temperature each time to the left side of the mannequins' hair.

Cosmetic Formula 6 was applied to the left side of a mannequin head using a spray bottle to fully saturate the hair, the left side of wet hair was then gently brushed for 25 minutes, after which it was rinsed for 3 min, and subsequently both sides of the hair were shampooed twice, then conditioned once, and finally let air dry. This Cosmetic Formula 6 application, brushing and rinsing treatment was performed once on one mannequin, twice on second mannequin with a week between each treatment, and thrice on a third mannequin with a week between each treatment. After completion of the Cosmetic Formula 6 treatment or repeated treatments, both sides of each mannequin head were washed by shampooing and subsequent conditioning five additional times, with the hair of each mannequin head fully air dried between each additional washing cycle.

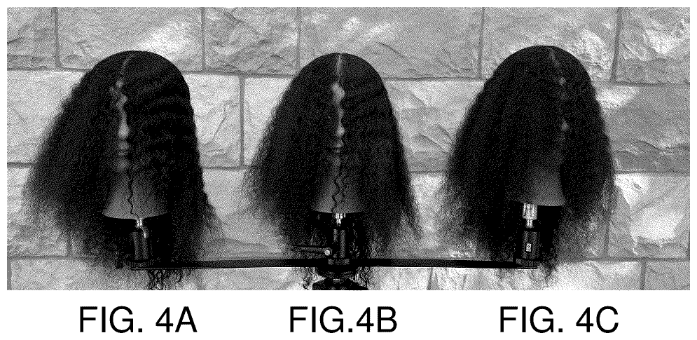

FIGS. 4A-4C show the final results of the treated left side of the mannequin's hair (i.e., the mannequin's left) and the control right side of the mannequin's hair (i.e., the mannequin's right), with the mannequin of FIG. 4A treated thrice, the mannequin of FIG. 4B treated twice, and the mannequin of FIG. 4C treated once. FIGS. 4A-4C demonstrate that Cosmetic Formulation 6 applied once, twice and thrice is able to increase smoothing and frizz management relative to the control, which is retained even after repeated washing. In addition, FIGS. 4A-4C demonstrate that repeated application of Cosmetic Formulation 6 showed increased smoothing and frizz management relative to hair with fewer treatments, even after repeated washing.

TABLE 6
Cosmetic Formulation 6

| Compounds | Amount (wt. %) |
|---|---|
| water | 77.4 |
| bis(2-ethylhexyl) maleate | 16.3 |
| ethanol | 1 |
| oleyl alcohol | 1 |
| oleic acid | 0.4 |
| stearamidopropyl dimethyl amine | 0.7 |
| sodium sulfite | 2.7 |
| vanillin | 0.5 |

### Example 6: Cosmetic Formulation vs. Control Formulation

A mannequin was treated with a sulfite solution of Control Formulation C₁ shown in Table 7 on the right side of the mannequin's hair (i.e., the mannequin's right), and compared to Cosmetic Formulation 6 applied to the left side of the mannequin's hair (i.e., the mannequin's left). Control Formulation C₁ was applied to the right side until the hair was saturated. Soon after, the hair was brushed for the 20 min, and subsequently washed by rinsing for 3 min, shampooing twice and conditioning once, with rinsing after each step in the washing process. After one week, this process was repeated.

On the left side of the mannequin, Cosmetic Formula 6 was applied to the left side until the hair was saturated. Soon after, the hair was brushed for 20 min, and subsequently washed by rinsing for 3 min, shampooing twice and conditioned once, with rinsing after each step in the washing process. After one week, the process was repeated.

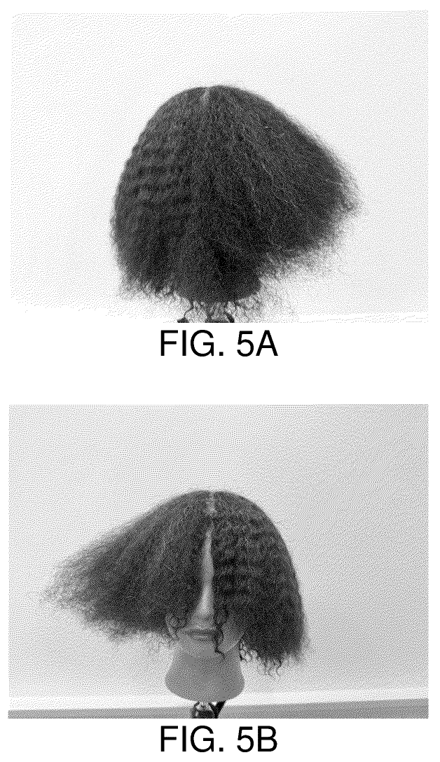

FIGS. 5A and 5B show the final results of the left side of the mannequin's hair (i.e., the mannequin's left) treated with the cosmetic formulation and the right side of the mannequin's hair (i.e., the mannequin's right) treated with a control formulation. The left side of the mannequin hair treated with the cosmetic formulation showed light relaxing of the hair with greatly reduced frizz, softer touch, increased shine and increased strength. In contrast, the right side of the mannequin hair treated with the control formulation showed light to moderate relaxing, but with increased frizz, harsh touch, decreased shine, increased brittleness when dry, and a spongy texture prone to easy breakage when wet.

TABLE 7
Control Formulation C1

| Compounds | Amount (wt. %) |
|---|---|
| water | 96.6 |
| sodium cocoyl isethionate | 0.2 |
| sodium sulfite | 2.9 |
| vanillin | 0.3 |

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount, depending on the desired function or desired result.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method of styling hair, comprising:
applying a cosmetic formulation to hair, wherein the cosmetic formulation comprises a compound of Formula (I) and a reducing agent comprising a sulfite salt, wherein the amount of compound of Formula (I) in the cosmetic formulation is at least about 1% by weight, and wherein the compound of Formula (I) has the structure: 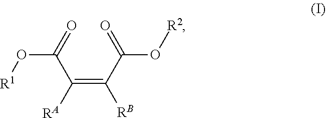
wherein:
each of R¹ and R² is independently selected from the group consisting of an optionally substituted C₈ alkyl; and
each of R^{A} and R^{B} is hydrogen; and
styling the hair;
wherein applying the cosmetic formulation and styling the hair achieves a hair style.

2. The method of claim 1, wherein styling the hair comprises contacting the hair with a styling tool.

3. The method of claim 1, wherein the styling tool is selected from the group consisting of a brush, a comb, a roller, and combinations thereof.

4. The method of claim 1, wherein the hair style is achieved at most about 30 minutes after the cosmetic formulation is applied to the hair.

5. The method of claim 1, wherein styling the hair is performed after applying the cosmetic formulation to the hair.

6. The method of claim 1, wherein applying the cosmetic formulation to the hair is performed after styling the hair.

7. The method of claim 1, wherein the hair style is substantially retained after washing the hair 1-50 times.

8. The method of claim 7, wherein washing the hair is selected from the group consisting of rinsing, shampooing, conditioning, drying, and combinations thereof.

9. The method of claim 1, wherein the cosmetic formulation is applied to the hair at least 1 time per month.

10. The method of claim 1, wherein the method provides increased hair smoothing, decreased hair frizz, improve touch, increased hair combability, increased hair shine, or combinations thereof.

11. The method of claim 1, wherein the hair style comprises permed hair, curled hair, straightened hair, relaxed hair, or combinations thereof.

12. The method of claim 11, wherein the method is a method of perming hair.

13. The method of claim 11, wherein the method is a method of straightening hair.

14. The method of claim 1, wherein the method does not comprise applying an oxidizing agent to the hair.

15. A cosmetic formulation, comprising:
about 0.1 wt. % to about 20 wt. % of a sulfite salt;
about 0.1 wt. % to about 20 wt. % of a surfactant;
a cosmetically acceptable carrier; and
about 0.1 wt. % to about 20 wt. % of a compound of Formula (I), wherein the compound of Formula (I) has the structure: 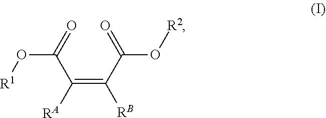
wherein:
each of R¹ and R² is independently selected from the group consisting of an optionally substituted C₈ alkyl; and
each of R^{A} and R^{B} is hydrogen.

16. The cosmetic formulation of claim 15, wherein the compound of Formula (I) is selected from the group consisting of dioctyl maleate and bis(2-ethylhexyl)maleate.

17. The cosmetic formulation of claim 15, wherein the cosmetic formulation comprises about 3 wt. % to about 18 wt. % of the compound of Formula (I).

18. The cosmetic formulation of claim 15, wherein the sulfite salt is selected from the group consisting of sodium sulfite, potassium sulfite, and a combination thereof.

19. The cosmetic formulation of claim 15, wherein the cosmetic formulation comprises about 0.8 wt. % to about 6 wt. % of the sulfite salt.

20. The cosmetic formulation of claim 15, wherein the surfactant comprises an anionic surfactant, a cationic surfactant, a natural surface active agent, or combinations thereof.

21. The cosmetic formulation of claim 20, wherein the surfactant comprises the anionic surfactant and the natural surface active agent.

22. The cosmetic formulation of claim 20, wherein the natural surface active agent comprises oleic acid.

23. The cosmetic formulation claim 20, wherein the anionic surfactant is selected from the group consisting of sodium cocoyl isethionate, stearamidopropyl dimethyl amine, oleyl alcohol, and combinations thereof.

24. The cosmetic formulation of claim 15, wherein the cosmetic formulation comprises about 0.5 wt. % to about 5 wt. % of the surfactant.

25. The cosmetic formulation of claim 15, wherein the cosmetically acceptable carrier comprises water, ethanol, or a combination thereof.

26. The cosmetic formulation of claim 15, wherein the cosmetic formulation comprises about 70 wt. % to about 95 wt. % of cosmetically acceptable carrier.

27. The cosmetic formulation of claim 15, wherein the cosmetic formulation has a pH of about 7.5 to about 10.

28. The cosmetic formulation of claim 15, further comprising a fragrance.

29. The cosmetic formulation of claim 28, wherein the cosmetic formulation comprises about 0.1 wt. % to about 1 wt. % of the fragrance.

30. A method of styling hair, comprising:
applying a cosmetic formulation to hair, wherein the cosmetic formulation comprises:
about 0.8 wt. % to about 6 wt. % of sodium sulfite;
about 0.5 wt. % to about 5 wt. % of a surfactant, wherein the surfactant comprises oleic acid, sodium cocoyl isethionate, stearamidopropyl dimethyl amine, oleyl alcohol, or combinations thereof;
about 3 wt. % to about 18 wt. % of bis(2-ethylhexyl)maleate;
about 0.1 wt. % to about 3 wt. % of ethanol; and
water;
wherein the cosmetic formulation is absent of an oxidizing agent; and
styling the hair;
wherein applying the cosmetic formulation and styling the hair achieves a hair style selected from the group consisting of permed hair, curled hair, straightened hair and relaxed hair, and combinations thereof; and
wherein applying the cosmetic formulation and styling the hair provides increased hair smoothing, decreased hair frizz, improved touch, increased hair combability, increased hair shine, or combinations thereof.
